(19) United States
(12) Patent Application Publication
(10) Pub. No.: US 2004/0072740 A1
(43) Pub. Date: Apr. 15, 2004

(54) Directed evolution of enzymes and antibodies

(75) Inventors: Brent Iverson, Austin, TX (US); George Georgiou, Austin, TX (US); Gang Chen, Austin, TX (US); Mark J. Olsen, Austin, TX (US); Patrick S. Daugherty, Austin, TX (US)

Correspondence Address: Steven L. Highlander, Esq., FULBRIGHT & JAWORSKI L.L.P., Suite 2400, 600 Congress Avenue, Austin, TX, 78701, US

(73) Assignee: Board of Regents, The University of Texas System.

(21) Appl. No.: 09/813,444

(22) Filed: Mar. 20, 2001

Related U.S. Application Data
(63) Continuation of application No. 08/847,063, filed on May 1, 1997, now abandoned.
Application No. 08/847,063 is a continuation-in-part of application No. 08/447,402, filed on May 23, 1995, now Pat. No. 5,866,344.
Application No. 08/447,402 is a continuation-in-part of application No. 08/258,543, filed on Jun. 10, 1994, now abandoned.
Application No. 08/258,543 is a division of application No. 07/794,731, filed on Nov. 15, 1991, now Pat. No. 5,348,867.

(51) Int. Cl. G01N 33/554; G01N 33/569; A61K 38/16

(52) U.S. Cl. 514/12; 435/7.32

(57) ABSTRACT

The invention relates to methods of selecting proteins, out of large libraries, having desirable characteristics. Exemplified are methods of expressing enzymes and antibodies on the surface of host cells and selecting for desired activities. These methods have the advantage of speed and ease of operation when compared with current methods. They also provide, without additional cloning, a source of significant quantities of the protein of interest.

The present application is a continuation-in-part of copending U.S. patent application Ser. No. 08/447,402, filed May 23, 1995, which is a continuation-in-part of U.S. Ser. No. 08/258,543, filed Jun. 10, 1994, which is a divisional of U.S. Pat. No. 5,348,867 issued Sep. 20, 1994. The entire text of each of the above-referenced applications and patents are specifically incorporated by reference herein without disclaimer.

The government owns rights in the present invention pursuant to grant number BCS-9412502 from the National Science Foundation.

### BACKGROUND OF THE INVENTION

1. Field of the Invention

The present invention relates generally to the fields of biochemistry, immunology and molecular biology. More particularly, it concerns the use of rapid selection techniques to identify specific polypeptides having desirable characteristics out of large libraries of polypeptides.

2. Description of Related Art

There is considerable interest in the discovery of new enzymes with catalytic and stability characteristics superior to what can be obtained from the pool of naturally-occurring enzymes in the biological world. For practical purposes, it is desirable to identify proteins that (i) can catalyze reactions (e.g., Diels-Alder condensation) that are important for chemical processing but do not occur in the biological world; (ii) exhibit unnatural chemical or stereoselectivity; (iii) can catalyze reactions in the presence of organic solvents or organic-aqueous mixtures that are typically used in chemical syntheses; and (iv) have an appreciable half-life at elevated temperatures and under other conditions of interest. Presently, methods for identifying these molecules are not readily available.

The development of enzymes with improved characteristics not only has important scientific ramifications, in terms of aiding the understanding of protein structure-function, but also is of great commercial value. In addition, most established pharmaceutical and biotechnology companies have extensive research efforts in place for enzyme engineering and optimization.

Antibodies also are of increasing importance in human therapy, assay procedures and diagnostic methods. Therefore, another area of interest lies in the identification of antibodies with particular binding functions, as well as other activities. However, methods of identifying antibodies and production of antibodies is often expensive, particularly where monoclonal antibodies are required. Hybridoma technology has traditionally been employed to produce monoclonal antibodies, but these methods are time-consuming and result in isolation and production of limited numbers of specific antibodies. Additionally, relatively small amounts of antibody are produced; consequently, hybridoma methods have not been developed for a large number of antibodies. This is unfortunate as the potential repertoire of immunoglobulins produced in an immunized animal is quite high, on the order of >10¹⁰, yet hybridoma technology is too complicated and time consuming to adequately screen and develop large number of useful antibodies.

Selective pressure, i.e., cell cultivation under conditions that allow growth only if a certain enzyme is active, has been used for many years to isolate mutants with desirable characteristics. The most successful approach is to mutagenize in vitro the gene for a desired enzyme, introduce the mutagenized DNA into cells to create a library and finally select for cells that produce active enzyme by growing under restrictive conditions. In one notable example, Liad et al. (1986) reported the isolation of thermostable variants of the E. coli kanamycin nucleotidyl transferase by introducing the gene in a thermophilic bacillus and selecting for mutants that could grow at a temperature above the inactivation temperature of the thermostable enzyme. More recently Palzkill et al. constructed sets of large libraries in which blocks of several residues in β-lactamase were randomized by in vitro techniques. The β-lactamase mutant genes were transformed into E. coli and cells capable of growing in the presence of β-lactam antibiotics that are normally poor β-lactamase substrates were isolated (Venkatachalam et al., 1994, Petrosino and Palzkill, 1996).

A variety of techniques including chemical mutagenesis of isolated DNA, gene amplification by error prone PCR™ and oligonucleotide mutagenesis have been employed to generate libraries of mutant genes containing a desired range of nucleotide substitutions. Often multiple rounds of selection and mutagenesis are employed to select increasingly improved enzymes. It is desirable to be able to combine the beneficial effects of mutations that exhibit an additive effect on function. For this purpose Stemmer (1994a and 1994b) devised a simple technique of DNA shuffling for allowing the combination of beneficial mutations in different parts of a gene. DNA shuffling is a powerful technique for the generation of combinatorial libraries in turn can allow drastic improvements in enzyme function. As demonstrated by Stemmer (1994a) DNA shuffling allowed a rapid isolation of a β-lactamase variant with a 32,000-fold higher activity towards the antibiotic cefotaxime.

The selection of improved enzymes from in vitro constructed libraries is a very powerful approach for biocatalyst design provided that the enzyme sought confers an essential function for the cell. Unfortunately in many cases of commercial interest it is not possible to design a selection strategy. In fact it is impossible to design selection strategies for reactions (such as Diels Alder condensation) that do not take place in biological systems. The other limitation of mutant selection strategies is that under selective conditions cells can evolve mechanisms of survival that bypass the reaction catalyzed by the enzyme that is to be optimized. The ability of cellular adaptation mechanisms to respond to selective pressure has frustrated efforts to direct the evolution of efficient antibody catalysts designed to complement mutations in essential pathways within microorganisms (Tang et al., 1991; Smiley and Benkovic, 1994).

For enzymatic reactions where the design of a selection strategy is not possible, libraries of mutants have to be screened by direct assay. This involves growing individual clones either as colonies on agar plates or, alternatively in 96-well plates, and measuring enzymatic activity usually by a chromogenic assay. A popular and convenient screening format is to determine the enzymatic activity of single colonies growing on agar plates. Sequential cycles of random mutagenesis and plate screening are increasingly employed for the directed evolution of enzyme activities. Using this approach Moore and Arnold (1996) isolated a mutant paranitrobenzyl esterase that exhibits 16-fold higher activity in 30% DMF relative to the parent enzyme. At least one other enzyme has been engineered successfully by random mutagenesis and screening (Yu and Arnold, 1996).

However, the isolation of clones producing desired enzymes by plate-screening is tedious and is not suitable when a drastic change in protein function or stability is sought. It is impractical to screen more than 10⁵ clones by plate assays (even with automated techniques) and therefore only a relative small set of mutants can be analyzed. Screening of much larger libraries of mutants, typically comprising of at least 1,000-fold larger number of clones (i.e., 10⁸) is necessary if there is to be any hope for completely changing enzyme activity or reversing its substrate specificity.

The screening of colonies using plate assays suffers form three additional limitations. First, plate assays can be devised for a limited range of reactions. Second, because the vast majority of proteins are not released from E. coli bacteria (by far the most preferred host organism for “directed evolution” studies), the substrate must be able to readily diffuse into the cell and it must not be toxic. Third, plate assays, even those that utilize fluorescent molecules have at best moderate specificity. In recent years there have been many efforts to find rapid assay screening methods that can circumvent the limitations inherent with plate assays. Perhaps the most innovative approach is the Catalytic ELISA (CatELISA) technique developed by Tawfik et al. (1993). However, neither CatELISA nor any of the other assay recent techniques can be applied to the screening of larger libraries of mutants.

Thus despite the intense interest in discovering new enzymes and antibodies, there remain a significant technical hurdles that have made it difficult to exploit this considerable wealth of biological power. Thus, there remains a great need for improved methods of handling the synthesis and identification of the vast number of possible active polypeptides.

### SUMMARY OF THE INVENTION

The present invention addresses these and other drawbacks inherent in the prior art by providing new methods of screening of polypeptide libraries. For the first time it is possible to rapidly screen polypeptide libraries for potential enzymes and antibodies; often in a matter of hours. The disclosed methods allow production of large quantities of these polypeptides, potentially on a kilogram scale, from microorganism cultures. And, because selected proteins can be displayed on the surface of a host cell, assays can be conducted with remarkable rapidity.

In one aspect of the invention, expression libraries are prepared such that an expressed protein is displayed on the surface of a cell. Typically, the polypeptides will be surface expressed in a host cell such as bacterial, yeast, insect, eukaryotic or mammalian cells. Surface expression of a polypeptide on a cell surface is achieved using a recombinant vector that promotes display on the outer membrane of a host cell. Vectors are such as those of the general construction described in U.S. Pat. No. 5,348,867, incorporated herein by reference. Generally the vectors will be appropriate for a bacterial host cell and will include at least three DNA segments as part of a chimeric gene. One segment is a DNA sequence encoding a polypeptide that targets and anchors a fusion polypeptide to a host cell outer membrane. A second DNA segment encodes a membrane-transversing amino acid sequence, i.e., a polypeptide that transports a heterologous or homologous polypeptide through the host cell outer membrane. The third DNA segment encodes any of a number of desired polypeptides. Such vectors will display fusion polypeptides at the exterior of a host cell. These recombinant vectors include a functional promoter sequence.

Screening for antibodies employing the methods of the present invention allows one to select an antibody or antibody fragment from a plurality of candidate antibodies that have been expressed on the surface of a host cell. In most instances the host cell will be a bacterial cell, preferably E. coli. The antibodies are obtained from an expression vector library that may be prepared from DNAs encoding antibodies or antibody fragments. One source of such DNAs could be from an animal immunized with a selected antigen; alternatively, antibody genes from other sources can be used, such as those produced by hybridomas or produced by mutagenesis of a known antibody gene. One preferred method of obtaining DNA segments is to isolate mRNA from antibody cells of an immunized animal. The mRNA may be amplified, for example by PCR, and used to prepare DNA segments to include in the vectors. One may also employ DNA segments that have been mutagenized from one or more DNAs that encode a selected antibody or antibody fragment.

In a second embodiment, the present invention provides methods for the rapid screening of enzyme libraries. The libraries represent mutagenized version of an enzyme to permit for the “directed” evolution of the enzyme's sequence, and hence function. Again, vectors will comprise a DNA sequence encoding the enzyme, an anchor fused to the enzyme coding region that results in expression of the host cell outer membrane and any other regulatory sequences necessary for the propagation of the vectors and the expression of the enzyme. Standard mutagenic procedures will be applied to various regions of the enzyme coding region, including those regions that encode binding pockets and active sites. Other sites of interest, including residues critical for conformation and post-translational modification also may be targeted.

Once generated, expression libraries are transferred, by standard methodologies, into appropriate host cells. Expression of the antibodies, enzymes or other polypeptides on the surface of host cells permits the rapid and efficient screening of libraries for the appropriate binding specificity, enzyme function or other desirable characteristic. In addition, use of appropriate label systems and substrates permits the sorting of host cells expressing proteins of interest by flow cytometery methodology (e.g., FACS).

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1A and FIG. 1B. FIG. 1A Western blot analysis of total membrane fractions from E. coli JM109 cells containing pTX101 (Lanes 2 and 4); JM109/pTX152 (Lanes 3 and 5) and probed with anti-OmpA antibodies at 1:5000 dilution; Lanes 4 and 5 were probed with monoclonal anti-HSV antibodies at 1:5000 dilution. Arrowheads indicate the Lpp-OmpA-β-lactamase fusion (lane 2) and the Lpp-OmpA-scFv(digoxin) fusion (lane 3). The 32 kDa band in lanes 2 and 3 corresponds to OmpA. Lane 1, molecular mass markers (in kDa). FIG. 1B. Lysate and whole cell ELISAs of JM109 cells containing plasmid pTX101 (solid) or pTX152 (hatched). Samples were incubated on microtiter wells coated with digoxin-conjugated BSA and probed with anti-β-lactamase (pTX101) or anti-HSV (pTX152) antibodies. Absorbance readings were referenced to wells that were untreated with either lysates or whole cells.

FIG. 2A and FIG. 2B. Phase contrast micrograph of JM109/pTX152 cells after 1 hr of incubation with 10⁻⁷ M digoxin-FITC. FIG. 2B. Micrographs of the same field as in FIG. 2A of JM109/pTX152 cells after a 1 hour incubation with 10⁻⁷M digoxin-FITC.

FIGS. 3A-3G. Histogram data from FACS. The bar in each graph represents the sorting gate or the fluorescence intensity defined as a positive event. The sorting gate was chosen to maximize the number of positive events while minimizing the number of negative events within the window. All samples were labeled with 10⁻⁷ M digoxin FITC. FIG. 3A. JM109/pTX152 sample used as a negative control. FIG. 3B. JM109/pTX152 sample used as a positive control. FIG. 3C. JM109/pTX152 pretreated with 0.2 mg/ml trypsin. FIG. 3D. JM109/pTX152 pretreated with free digoxin. FIG. 3E. A 100,000:1 mixture of JM109/pTX101:JM109/pTX152 prior to the first cell sorting run. FIG. 3F. A 100,000:1 mixture after growing cells recovered from the first cell sorting run. FIG. 3G. A 100,000:1 mixture after growing cells recovered from the second cell sorting run.

FIG. 4. Whole cell immununoassay using 0.5 nM FITC labeled digoxin.

FIGS. 5A and 5B: FIG. 5A. Antibody mutants displaying different affinity for the antigen can be distinguished by display on the cell surface and fluorescence activated cell sorting. A. Fluorescence histogram comparing the fluorescence distribution of bacterial cells displaying mutants of the svFv (digoxin) antibody on their surface. FIG. 5B. Relative binding affinity of the corresponding purified antibodies measured by ELISA.

FIGS. 6A and 6B Immunoassay for the determination of the equilibrium constant for antigen binding for surface displayed scFv antibodies. Cells displaying scFv(digoxin) antibodies on their surface were incubated with different concentrations of BODIPY-digoxin for one hour with gentle shaking. Following incubation, the fluorescence distribution of the cells (50,000 events) was determined by flow cytometry.

FIG. 7: Single chain antibody surface display plasmid vector pSD192

FIG. 8: Procedure for the isolation of single-chain antibodies by surface display and FACS.

FIGS. 9A-9F: Isolation of high affinity antibodies from libraries displayed on the bacterial cell surface and screened by FACS. FIGS. 9A-9C: Plots of Forward Scatter (a measure of the cell size) as a function of fluorescence intensity for cell populations displaying a library of scFv antibodies and incubated with different concentrations of fluorescent hapten (BODIPY-digoxin). The library was constructed by randomizing the heavy chain residues 99, 100, 100a and 100b as described in Example 7. Cells were incubated with Bodipy-digoxin at different concentrations, shown in FIGS. 9A-9C, for one hour. Each point represents one event detected by the flow cytometer and a total of 50,000 events (i.e. cells) are shown for clarity. FIGS. 9D-9F. Isolation of scFv antibodies from a library. The library was constructed by randomizing the heavy chain residues 99, 100, 100a and 100b as described in Example 7. Cells were incubated with 70 nM BODIPY-digoxin and high fluorescence clones were isolated by FACS. The corresponding fluorescence histogram is shown in FIG. 9D. Sorted cells were grown overnight, incubated with 15 nM BODIPY-digoxin and sorted. The fluorescence distribution of the cells is shown in FIG. 9E. As can be seen, after one round of sorting and growth, cells having high fluorescence are greatly enriched over the starting cell population. Finally, FIG. 9F shows the fluorescence distribution of the cell population obtained after growing the positive cells isolated in the first round. The large majority of the cells bind the BODIPY-digoxin conjugate and thus have a high fluorescence.

FIG. 10: Chemical Structure of the OmpT substrate

FIG. 11A and FIG. 11B: FIG. 11A shows fluorescence histograms of cells expressing inactive OmpT after exposure to the fluorogenic substrate. FIG. 11B shows the flow cytometric fluorescence of cells expressing wild-type (active) OmpT) after exposure to the fluorogenic substrate.

FIGS. 12A-12C: FIG. 12A shows a fluorescence histogram of 20,000 cells from a mixture of OmpT⁺ and OmpT⁻ at a ratio of 1:5,000. After sorting, 32 cells were collected and the fluorescence of nine clones was examined by FACS. FIG. 12B and FIG. 12C show representative fluorescence histograms for two of the isolated OmpT+ clones.

### DESCRIPTION OF ILLUSTRATIVE EMBODIMENTS

A. Identifying Polypeptides With Desirable Properties

As stated above, the natural diversity of biologically active polypeptides, in terms of both function and specificity, is immense. The problem lies in tapping this vast reservoir of potential reagents in a sufficiently expedient fashion. In other words, how does one identify the few polypeptides out of the almost infinite number of possibilities that function as desired? Automated selection technologies may speed up the process, but the real obstacle remains the sheer number of possibilities—how they can be generated and how they can be expressed in order for the selection to take place. The present invention is designed to address this particular problem, as described in greater detail below.

Enzymes

Efforts to engineer improved enzymes rely upon molecular biology techniques and involve two basic approaches. The first involves cloning and expression of enzyme libraries from organisms that cannot be cultivated and typically are isolated from extreme environments. This approach, pioneered by Recombinant Biocatalysis (Brennan, 1996), relies on the construction expression libraries by extracting DNA from samples and gene amplification by PCR™. The expression libraries are then screened by brute force approaches that rely heavily on robotics. This technology promises to begin to tap the unexplored diversity of function in the natural world. However, it is intrinsically limited to catalytic activities that serve a biological function. Also, it is limited by the ability to employ genes from unknown sources to direct the synthesis of functional properties in commonly used host organisms such as E. coli or Saccharomyces cerevisiae.

A second approach relies on selecting antibodies with the appropriate enzymatic or catalytic characteristics. The principle underlying catalytic antibodies is straightforward—binding of an antibody to its cognizant antigen results in a free energy decrease. An antibody that is highly complementary to the rate-limiting species of a chemical reaction (i.e., the transition state complex) will lower the energy of that species. This decrease in the free energy of formation of the transition state translates into a higher rate of reaction. In general terms, catalytic antibodies are produced via immunization with a molecule (a hapten) that is designed to mimic certain features of the transition state complex for the reaction of interest (Lerner et al., 1991). An impressive array of different reactions now have been catalyzed by antibodies. Moreover, as expected for a catalyst based on antibody recognition, catalytic antibodies display precise substrate selectivity. That is, only substrates that are similar in structure to the hapten used to elicit the catalytic antibodies are accepted in the catalytic reaction.

Unfortunately, catalytic antibodies have not yet fulfilled initial expectations. A number of reasons are at the root of the slow progression. First, the generation of catalytic antibodies is technically difficult and prohibitively expensive. Second, production costs are uneconomical. Despite impressive advances in hybridoma culture scale-up, the cost-effective production of monoclonal antibodies remains a serious challenge. Third, poor kinetic properties are common. Typically, rates of reaction and acceleration with catalytic antibodies (i.e., rate of catalyzed reaction over uncatalyzed reaction) are between 10⁴-10⁵, although a few examples of higher rates have been reported (Janda et al., 1988; however also see Hollfelder et al., 1996). For comparison, rates of reaction acceleration for enzymes are usually around 10⁷-10⁸. Fourth, transition state mimics must be immunogenic and stable in vivo. Animal immunization with the transition state analog is the first step in the production of monoclonal antibodies with catalytic activity. The need for immunization poses two serious constraints on the transition state analog; it must be recognized by the immune response and it must be stable in the animal.

Antibodies

Currently, the most widely used approach for screening polypeptide libraries is to display polypeptides on the surface of filamentous bacteriophage (Smith, 1991; Smith, 1992). The polypeptides are expressed as fusions to the N-terminus of a coat protein. As the phage assembles, the fusion proteins are incorporated in the viral coat so that the polypeptides become displayed on the bacteriophage surface. Each polypeptide produced is displayed on the surface of one or more of the bacteriophage particles and subsequently tested for specific ligand interactions. While this approach appears attractive, there are numerous problems, including difficulties of enriching positive clones from phage libraries. Enrichment procedures are based on selective binding and elution onto a solid surface such as an immobilized receptor. Unfortunately, avidity effects arise due to multivalent binding of the phage and the general tendency of phage to contain two or more copies of the displayed polypeptide. The binding to the receptor surface therefore does not depend solely on the strength of interaction between the receptor and the displayed polypeptide. This causes difficulties in the identification of clones with high affinity for the receptor; thus, there remain distinct deficiencies in the methods used to isolate and screen polypeptides, particularly antibodies, even in view of the development of phage libraries.

Previous Attempts to Develop Engineered Polypeptides

An approach to the antibody selection problem has been the development of library screening methods for the isolation of antibodies (Huse et al, 1989; McCafferty et al., 1990; Chiswell & McCafferty, 1992; Chiswell & Clackson, 1992; Clackson, 1991). Functional antibody fragments have been produced in E. coli cells (Skerra & Pluckthun, 1988; Better et al., 1988; Orlandi et al., 1989; Sastry et al., 1989) as “libraries” of recombinant immunoglobulins containing both heavy and light variable domains (Huse et al, 1989). The expressed proteins have antigen-binding affinity comparable to the corresponding natural antibodies. However, it is difficult to isolate high binding populations of antibodies from such libraries and where bacterial cells are used to express specific antibodies, isolation and purification procedures are usually complex and time-consuming.

Combinatorial antibody libraries generated from phage lambda (Huse et al, 1989) typically include millions of genes of different antibodies but require complex procedures to screen the library for a selected clone. Ladner et al., (U.S. Pat. No. 5,403,484, specifically incorporated herein by reference) reported the display of proteins on the outer surface of a chosen bacterial cell, spore or phage, in order to identify and characterize binding proteins. Certain elements of Ladner may be used advantageously, for example, methods of generating and expressing single chain antibodies, proteinaceous binding domains other than a single chain antibody, carrier protein and the like, in combination with the present invention.

Methods have been reported for the production of human antibodies using the combinatorial library approach in filamentous bacteriophage. A major disadvantage of such methods is the need to rely on initial isolation of the antibody DNA from peripheral human blood to prepare the library.

One way of approaching enzyme selection relies on the possibility of obtaining accurate 3-dimensional structures for polypeptides, which then can be employed to identify amino acid substitutions that can alter or enhance function. Structure-guided mutagenesis in protein chemistry is best exemplified by the elegant and extensive studies of Matthews and coworkers using the T4 lysozyme as a model (Matthews, 1995). Over the years his group constructed, characterized and solved the crystal structure of over 100 T4 mutants. These studies have provided valuable information on the determinants of protein stability. A few mutants with markedly increased thermostability have been isolated. However, structure guided mutagenesis is not likely to become a general route to the isolation of new biocatalysts because, (i) the three dimensional structure of a protein is required as a starting point, and the three dimensional structure is not available for most polypeptides and (ii) while the prediction of amino acid substitutions that affect stability has been met with some success, mutations that increase or dramatically alter catalytic activity have been very difficult to predict.

B. The Present Invention

It is proposed, according to the present invention, to provide a general approach for the efficient screening of very large libraries of virtually any polypeptide for desirable activity. This approach employs directed evolution for the selection of enzymes and, further, can be applied to the screening of antibody libraries. This provides an extremely powerful tool for the selection of such polypeptides. Moreover, the development of methods that achieve stable anchoring and polypeptide display on the surface of a bacterial cell such as E. coli, has provided the basis for new methods of rapid and efficient selection of polypeptides from libraries expressed in the host cells. With the polypeptide on the surface, exposed directly to the aqueous media, the activity of the polypeptide can be assayed directly, without having to worry about transport of assay reagents into cells.

The methods disclosed herein are particularly advantageous because they allow unprecedented rapid and efficient selection, purification and screening of polypeptide libraries from bacterial host cell surfaces, providing several advantages over phage libraries. Unlike most other methods used for screening and assay, the disclosed methods are well-suited for commercial adaptation. Assay procedures are greatly facilitated because of the cell surface display aspect, permitting the use of simple centrifugation to remove the cells from an assay sample. Assays thus are very rapid and inexpensive as they do not require complex or expensive equipment.

In a first embodiment, relating to directed enzyme evolution, the invention comprises the following approach. A target enzyme (or catalytic antibody) which is to be subjected to mutagenesis is first displayed on the surface of E. coli bacteria as a fusion to a surface-targeting vehicle. This technology was developed by Georgiou and coworkers and has been used to display a number of proteins on the bacterial surface (Francisco et al., 1992; 1993a; 1993b; Georgiou et al., 1996). By displaying the target enzyme on the bacterial surface it will be fully accessible to molecules in the extracellular fluid. Thus, the enzyme is free to react with any substrate added to the cells without any of the limitations that are imposed when intracellular enzymes are studied.

Expression of recombinant proteins on cell surfaces of Gram-negative bacteria is achieved by fusion to segments of a major lipoprotein and OmpA; however, fusion to protein domains other than those derived from the major lipoprotein and OmpA is also envisioned, provided that these domains can function for the expression of the desired polypeptide on the cell surface. Generally, the desired polypeptide is fused to an amino acid sequence that includes the signals for localization to the outer membrane and for translocation across the outer membrane. The amino acid sequences responsible for localization and for translocation across the outer membrane may be derived either from the same bacterial protein or from different proteins of the same or different bacterial species. Examples of proteins that may serve as sources of localization signal domains are shown in Table 1.

TABLE 1
EXAMPLES OF OUTER MEMBRANE TARGETING SEQUENCES

| | Organism |
|---|---|
| Lpp | E. coli (or functional equivalent in |
| | Salmonella) |
| TraT | E. coli (or functional equivalent in |
| | Salmonella) |
| OsmB | E. coli (or functional equivalent in |
| | Salmonella) |
| N1pB | E. coli (or functional equivalent in |
| | Salmonella) |
| BIaZ | E. coli (or functional equivalent in |
| | Salmonella) |
| Lpp1 | Pseudomonas aeruginosa |
| PA1 | Haemophdus influenza |
| OprI | E. coli |
| 17 kDa 1pp | Riokettsia riokettsii |
| H.8 protein | Neisseria gonorrhea |

In addition a sequence that allows display of the polypeptide on the cell surface is required. Appropriate examples are shown in Table 2.

TABLE 2
EXAMPLES OF TRANSMEMBRANE SEQUENCES

| | |
|---|---|
| OmpA | E. coli or functional equivalent in Salmonella |
| LamB | E. coli or functional equivalent in Salmonella |
| PhoE | E. ccli or functional equivalent in Salmonella |
| OmpC | E. coli or functional equivalent in Salmonella |
| OmpF | E. coli or functional equivalent in Salmonella |
| OmpT | E. coli or functional equivalent in Salmonella |
| FepA | E. coli or functional equivalent in Salmonella |

Several fusion protein strategies for the display of relatively short peptides on the surface of Gram-negative bacteria have been described (Table 3). Short peptides of less than 60 amino acids residues can be displayed on the cell surface when fused into surface exposed loops of outer membrane proteins (OMPs) from enteric bacteria (Hofnung et al., 1991; Charbitt et al., 1988; Agterberg et al. 1990; Su et al., 1992; Wong et al., 1995; Newton et al., 1996). Hofnung and coworkers were the first to demonstrate that peptides inserted within permissive sites of the E. coli outer membrane protein LamB are displayed on the cell surface, accessible to antibodies in the extracellular fluid and have thus been exploited extensively for practical applications Hofnung et al., 1991; Brown, 1992; O'Callaghan et al., 1990; Sousa et al, 1996). However, it was quickly realized that the insertion of peptides longer than 60 amino acids perturbs the overall conformation and assembly of the carrier, interfering with the localization of the fusion proteins (Hofnung et al., 1991; Charbitt et al., 1988; Agterberg et al. 1990). Moreover, the positioning and length of the peptide insert plays a critical role in the efficient surface display and recognition of the inserted epitope (Su et al., 1992; Wong et al., 1995; Newton et al., 1996).

TABLE 3
Expression Systems for protein display in E. coli

| Carrier | Type of Fusion | Localization of Passenger | Passenger Polypeptide | Applications |
|---|---|---|---|---|
| E. coli LamB | sandwich fusion | cell surface | variety of viral peptide | vaccines, peptide libraries, |
| | | | antigen | cellular absorbants |
| E. coli PhoE | sandwich fusion | cell surface | epitope from hsp65 of M. | vaccines |
| | | | tuberculosis | |
| Pseudomonas OprF | sandwich fusion | cell surface | 4 aa epitope from malaria | vaccines |
| | | | parasite | |
| E. coli or other | C-terminal or | periplasmic side of outer | scFv antibodies; 11 aa CE | lipid tagged antibodies, |
| Gram-negative | sandwich fusions | membrane/cell surface | epitope of polio virus | vaccines |
| lipoprotein | | | | |
| E. coli Lpp-OmpA | C-terminal fusion | cell surface | scFv antibodies; β- | peptide/antibody libraries, |
| | | | lactamase; protein A; | cellular adsorbents, |
| | | | cellulose binding protein | immunoassays |
| Shigella VirG₈ | N-terminal fusion | cell surface | alkaline phosphatase | |
| Neisseria IgA₈ | N-terminal fusion | cell surface | cholera toxin B subunit | vaccines peptide libraries |
| E. coli Flagellin | sandwich fusion | cell surface | thioredoxin; peptides | peptide libraries |
| (Flic) | | | inserted within thioredoxin | |
| Salmonella | sandwich fusion | cell surface | 18 aa epitope from HIV1 | vaccines |
| Flagellin (FliC) | | | gp41 protein | |
| E. coli FimH (Type | sandwich fusion | cell surface | 52 aa sequence from the | vaccines |
| I pili) | | | preS2 hepatitis B antigen | |
| E. coli PapA | sandwich fusion | cell surface | 58 aa domain from | cellular adsorbents |
| | | | Staphylococcus protein A | |
| Klebsiella PulA | C-terminal fusion | cell surface/extracellular | B-lactamase | |
| | | fluid | | |

In a different approach, Fiers and colleagues expressed the immunoglobulin G-binding domain of protein A of Staphylococcus aureus on the surface of E. coli using PapA, the major subunit of the Pap pilus (Steidler et al., 1993). Other groups have used flagellum or pilus subunits to develop expression systems for the surface presentation of antigenic/immunogenic epitopes derived from pathogens, suitable for the development of live recombinant vaccines (Newton et al., 1995; Pallesen et al., 1995; Van Die et al., 1990).

For reasons that are not fully understood, subunits of cellular appendages and outer membrane porins are not suitable for the surface display of large polypeptides. To overcome this problem it has been necessary to use surface display carrier proteins that are exported via more specialized mechanisms (Salmong et al., 1993). For example, the targeting of many lipoproteins from Gram-negative bacteria onto the outer membrane is determined only by the presence of a short N-terminal sequence. Because of this property, several lipoproteins have been tested as potential carriers for surface display (Taylor et al., 1990; Laukkanen et al., 1993; Fuchs et al., 1991; Cornellis et al., 1996). Unfortunately, lipoprotein fusions have been found to be either detrimental to the integrity of the cell envelope causing extensive cell lysis, or to be tethered to the interior face of the outer membrane, in which case they are not exposed to the extracellular fluid (Laukkanen et al., 1993; Comellis et al., 1996).

These limitations have been addressed by constructing an Lpp-OmpA hybrid display vehicle consisting of the N-terminal outer membrane localization signal from the major lipoprotein (Lpp) fused to a domain from the outer membrane protein OmpA (Franscisco et al., 1992). OmpA mediates the display of passenger proteins fused to the C-terminal of the Lpp-OmpA hybrid. Lpp-OmpA fusions have been used to successfully display on the surface of E. coli several proteins varying in size between 20 and 54 kDa (Stathopoulos et al., 1996). Among the proteins that have been tested thus far only the dimeric bacterial enzyme alkaline phosphatase (phoA) could not be displayed on the cell surface (Stathopoulos et al., 1996).

The IgA proteases of Neisseria gonorrhoeae and Hemophilus influenzae use a variation of the most common, Type II secretion pathway (Salmong et al., 1993), to achieve extracellular export independent of any other gene products (Klauser et al., 1993). Specifically, the C-terminal domain of the IgA protease forms a channel in the outer membrane that mediates the export of the N-terminal domain across the membrane which in turn becomes transiently displayed on the external surface of the bacteria. This export mechanism is used by a number of extracellular proteins from pathogenic bacteria (Klauser et al., 1993; Jose et al., 1995; Suzuki et al., 1995; Provence et al., 1997; St. Geme et al., 1994). Replacement of the native N-terminal domain of IgA protease or VirG with the cholera toxin B subunit or the periplasmic E. colic protein MalE, respectively, resulted in the surface presentation of the passenger polypeptides (Suzuki et al., 1995; Klauser et al., 1990).

Unlike the IgA protease, the lipoprotein pullulanase (PulA) of Klebsiella pneumoniae, which is also exported via a type Ii secretion mechanism, requires 14 genes for its translocation across the outer membrane (Salmong et al., 1993). Pugsley and coworkers have shown that the lipoprotein pullulanase (PulA) can facilitate translocation of the periplasmic enzyme β-lactamase across the outer membrane. However, pullulanase hybrids remain only temporarily attached to the bacterial surface and are subsequently released into the medium (Kornacker et al., 1990). Although the lack of permanent association with the cell wall is not detrimental for vaccine development, it is a serious limitation in other applications such as library screening.

Expression systems for the display of proteins in Gram-positive bacteria have also been developed (Fischetti, 1996). Uhlen and colleagues used fusions to the cell-wall bound, X-domain of protein A, for the display of foreign peptides up to 88 amino acids long to the surface of Staphylococcus strains (Hansson et al., 1992; Samuelson et al., 1995). In other studies, the fibrillar M6 protein of Streplococcus pyogenes was employed as a carrier for antigen deliver in Streptococcus cells (Pozzi et al., 1992).

Protein display applications has also spurred the development of suitable expression systems for yeast cells. Surface display expression systems for yeast have relied primarily on the fusion of passenger proteins to agglutinin, a protein involved in cell adhesion (Schreuder et al., 1993; Schreuder et al., 1996; Schreuder et al., 1996; Boder and Wittrup et al 1996). The AGα1 agglutinin is tightly bound to the cell wall through its C-terminus. N-terminal fusions to the cell wall domain of AGA1 are stably anchored on the cell surface. This system has been used for the surface expression of a variety of enzymes and binding proteins (Schreuder et al., 1996). Mating-type a cells use the two subunit agglutinin a for cell adhesion. Recently the second subunit of agglutinin a (Aga2p) was used as a vehicle for the surface display of antibodies and peptides (Boder and Wittrup et al., 1996). In this case, the passenger polypeptide is fused to the C-terminus of AGA2 which, in turn, is linked to the AGA1 via disulfide bonds.

The gene encoding the target enzyme is mutagenized by conventional techniques to generate a library of mutants. The library of mutants will be screened using a highly sensitive single cell assay. Cells exhibiting the desired activity will be isolated. This will involve the following: (i) design of a fluorescent substrate for the desired reaction; and may involve immobilization of the cells onto micron-size particles; and (ii) screening and isolation of fluorescent rnicroparticles either by fluorescent activated cell sorting or by using a micromanipulator.

In a second embodiment, the present invention relies on the same cell surface display of polypeptides as described above, except that the polypeptide to be expressed on the host cell surface is an antibody. The screening methods are practiced by first constructing an antibody library, using any of several well-known techniques for library construction. For example, after selecting an immunogen, one may immunize a mammal by conventional means and collect antiserum. mRNA from spleen may be used as template for PCR amplification; for example employing primers complementary to constant and variable domain framework regions of different antibody subclasses. Alternatively, DNA from polyclonal populations of antibodies may be amplified, fragmented if desired, ligated into pTX101 or a similar vector as described in U.S. Pat. No. 5,348,867, incorporated herein by reference, and transformed into a host cell.

C. Expression Libraries and Mutagenesis

The present invention involves the display of polypeptides on the surface of a bacteria. U.S. Pat. No. 5,348,867, addressing this topic, is specifically incorporated by reference. As used herein, the term polypeptide refers to any protein and includes antibodies, antibody fragments, receptors, enzymes, cytokines, transcription factors, clotting factors, chelating agents and hormones.

Genes for polypeptides of interest are fused to the 3′ of a sequence that encodes a cell surface targeting domain. The cell envelope of E. coli and other gram-negative bacteria consists of the inner membrane (cytoplasmic membrane), the peptidoglycan cell wall and the outer membrane. Although the latter normally serves as a barrier to protein secretion, a targeting sequence has been developed that, when fused to normally soluble proteins, can direct them to the cell surface (Francisco et al., 1992; 1993). The surface targeting domain includes the first nine amino acids of Lpp, the major lipoprotein of E. coli fused to amino acid 46-159 of the Outer Membrane Protein A (OmpA). The function of the former is to direct the chimera to the outer membrane whereas the OmpA sequence mediates the display of proteins at the C-terminal of OmpA. Lpp-OmpA(46-159) fusions have been used to anchor a variety of proteins such as β-lactamase, a cellulose binding protein and alkaline phosphatase on the E. coli surface. However, other analogous surface targeting domains may be employed to stably anchor the recombinant polypeptides on the cell surface.

Methods for the display of virtually any polypeptide on the surface E. coli are described in U.S. Pat. No. 5,348,867. Any library encoding a set of related polypeptide sequences may be displayed on the surface of E. coli. Examples of such libraries include, libraries derived by mutagenesis of a homologous or heterologous protein, random peptide libraries, epitope libraries and libraries of recombinant antibody fragments, all of which may be created by methods known to those skilled in the art.

Exemplary of the surface expression method is an Lpp-OmpA (46-159)-antibody fusion expressed in a gram-negative bacterium. Due to the presence of the Lpp-OmpA(46-159) sequence, the fusion is localized on the outer membrane such that the N-terminal domain is embedded in the bilayer and the antibody sequence is fully exposed on the cell surface. Recombinant antibodies expressed on the cell surface as Lpp-OmpA(46-159) fusions are functional and bind to antigens with high affinity. Such fusions, manipulated as described below, will constitute preferred forms of the expression libraries of the present invention.

Mutagenesis

Where employed (i.e., directed evolution), mutagenesis will be accomplished by a variety of standard, random mutagenic procedures. Mutation is the process whereby changes occur in the quantity or structure of an organism. Mutation can involve modification of the nucleotide sequence of a single gene, blocks of genes or whole chromosome. Changes in single genes may be the consequence of point mutations which involve the removal addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They are also induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of bas e sequence when the affected DNA is replicated or repaired and thus to a mutation.

Insertional Mutagenesis

Insertional mutagenesis is based on the inactivation of a gene via insertion of a known DNA fragment. Because it involves the insertion of some type of DNA fragment, the mutations generated are generally loss-of-function rather than gain-of-function mutations. However there are several examples of insertions generating gain-of-function mutations (Moncz et al. 1990; Oppenheimer et al. 1991). Insertion mutagenesis has been very successful in bacteria and Drosophila (Cooley et al. 1988) and recently has become a powerful tool in several plant species (corn; e.g., Schmidt et al. 1987); Arabidopsis; e.g., Herman and Marks 1989; Koncz et al. 1990; Antirrhinum: e.g., Sommer et al. 1990).

Transposable genetic elements are DNA sequences that can move (transpose) from one place to another in the genome of a cell. The first transposable elements to be recognized were the Activator/Dissociation elements of Zea mays (McClintock, 1957). Since then they have been identified in a wide range of organisms, both prokaryotic and eukaryotic (Berg and Howe 1989).

Transposable elements in the genome are characterized by being flanked by direct repeats of a short sequence of DNA that has been duplicated during transposition and is called a target site duplication. Virtually all transposable elements whatever their type, and mechanism of transposition, make such duplications at the site of their insertion. In some cases the number of bases duplicated is constant, in other cases it may vary with each transposition event. Most transposable elements have inverted repeat sequences at their termini. these terminal inverted repeats may be anything from a few bases to a few hundred bases long and in many cases they are known to be necessary for transposition.

Prokaryotic transposable elements have been most studied in E. coli and Gram negative bacteria but are also present in Gram positive bacteria. They are generally termed insertion sequences if they are less than about 2 kilobases long or transposons if they are longer. Bacteriophages such as mu and D108 which replicate by transposition make up a third type of transposable element. elements of each type encode at least one polypeptide a transposase, required for their own transposition. Transposons further often include genes coding for function unrelated to transposition and often carry antibiotic resistance genes.

Transposons can be divided into two classes according to their structure. Firstly, compound or composite transposons have copies of an insertion sequence element at each end usually in an inverted orientation. These transposons require transposases to be encoded by one of their terminal IS elements. The second class of transposon have terminal repeats of about 30 base pairs and do not contain sequences from IS elements.

Transposition is usually either conservative or replicative although in some cases it can be both. In replicative transposition one copy of the transposing element remains at the donor site and another is inserted at the target site. Conservative transposition the transposing element is excised from one site and inserted at another.

Eukaryotic elements can also be classified according to their structure and mechanism of transportation. The primary distinction is between elements that transpose via an RNA intermediate and elements that transpose directly from DNA to DNA.

Elements that transpose via an RNA intermediate are often referred to as retrotransposons and their most characteristic feature is that they encode polypeptides that are believed to have reverse transcriptionase activity. There are two types of retrotransposon: some resemble the integrated proviral DNA of a retrovirus in that they have long direct repeat sequences, long terminal repeats (LTRs), at each end. The similarity between these retrotransposons and proviruses extends to their coding capacity. They contain sequences related to the gag and pol genes of a retrovirus, suggesting that they transpose by a mechanism related to a retroviral life cycle. Retrotransposons of the second type have no terminal repeats. They also code for gag- and pol-like polypeptides and transpose by reverse transcription of RNA intermediates but do so by a mechanism that differs from that or retrovirus-like elements. Transposition by reverse transcription is a replicative process and does not require excision of an element from a donor site.

Transposable elements are an important source of spontaneous mutations and must have influenced the ways in which genes and genomes have evolved. They can inactivate genes by inserting within them and can cause gross chromosomal rearrangements either directly through the activity of their transposases or indirectly as a result of recombination between copies of an element scattered around the genome. Transposable elements that excise often do so imprecisely and may produce alleles coding for altered gene products if the number of bases added or deleted is a multiple of three.

Transposable elements themselves may evolve in unusual ways. If they were inherited like other DNA sequences then copies of an element in one species would be more like copies in closely related species than copies in more distant species. This is not always the case, suggesting that transposable elements are occasionally transmitted horizontally from one species to another.

Chemical Mutagenesis

Chemical mutagenesis offers certain advantages, the ability to find a full range of mutant alleles with degrees of phenotypic severity, and is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the O4 position of thymine induced by exposure to n-nitrosoureas results in TA to CG transitions

A high correlation between mutagenicity and carcinogenity is the underlying assumption behind the Ames test (McCann et al., 1975 incorporated herein by reference) which speedily assays for mutants in a bacterial system, together with an added rat liver homogenate, which contains the microsomal cytochrome P450, to provide the metabolic activation of the mutagens where needed.

In vertebrates several carcinogens have been found to produce mutation in the ras proto-oncogene. N-nitroso-N-methyl urea induces mammary, prostate and other carcinomas in rats with the majority of the tumors showing a G to A transition at the second position in codon 12 of the Ha-ras oncogene. Benzo[a]pyrene-induced skin tumors contain A to T transformation in the second codon of the Ha-ras gene.

Radiation Mutagenesis

The integrity of biological molecules is degraded by the ionizing radiation. Adsorption of the incident energy leads to the formation of ions and free radicals, and breakage of some covalent bonds. Susceptibility to radiation damage appears quite variable between molecules, and between different crystalline forms of the same molecule. It depends on the total accumulated dose, and also the dose rate (as once free radicals are present, the molecular damage they cause depends on their natural diffusion rate and thus upon real time). Damage is reduced and controlled by making the sample as cold as possible.

Ionizing radiation causes DNA damage and cell killing, generally proportional to the dose rate. Ionizing radiation has been postulated to induce multiple biological effects by direct interaction with DNA or through the formation of free radical species leading to DNA damage (Hall, 1988). These effects include gene mutations, malignant transformation, and cell killing. Although ionizing radiation has been demonstrated to induce expression of certain DNA repair genes in some prokaryotic and lower eukaryotic cells, little is known about the effects of ionizing radiation on the regulation of mammalian gene expression (Borek, 1985). Several studies have described changes in the pattern of protein synthesis observed after irradiation of mammalian cells. For example, ionizing radiation treatment of human malignant melanoma cells is associated with induction of several unidentified proteins (Boothman, et al., 1989). Synthesis of cyclin and co-regulated polypeptides is suppressed by ionizing radiation in rat REF52 cells but not in oncogene-transformed REF52 cell lines (Lambert and Borek, 1988). Other studies have demonstrated that certain growth factors or cytokines may be involved in x-ray-induced DNA damage. In this regard, platelet-derived growth factor is released from endothelial cells after irradiation (Witte, et al., 1989).

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an x-radiation. The amount of ionizing radiation needed in a given cell generally depends upon the nature of that cell. Typically, an effective expression-inducing dose is less than a dose of ionizing radiation that causes cell damage or death directly. Means for determining an effective amount of radiation are well known in the art.

In a certain embodiments, an effective expression inducing amount is from about 2 to about 30 Gray (Gy) administered at a rate of from about 0.5 to about 2 Gy/minute. Even more preferably, an effective expression inducing amount of ionizing radiation is from about 5 to about 15 Gy. In other embodiments, doses of 2-9 Gy are used in single doses. An effective dose of ionizing radiation may be from 10 to 100 Gy, with 15 to 75 Gy being preferred, and 20 to 50 Gy being more preferred.

Any suitable means for delivering radiation to a tissue may be employed in the present invention in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

Random and In Vitro Scanning Mutagenesis

Random mutagenesis may also be introduced using error prone PCR (Cadwell and Joyce, 1992). The rate of mutagenesis may be increased by performing PCR in multiple tubes with dilutions of templates.

Structure-guided site-specific mutagenesis represents a powerful tool for the dissection and engineering of protein-ligand interactions (Wells, 1996, Braisted and Wells, 1996). One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et al., 1989).

Comprehensive information on the functional significance and information content of a given residue of an antibody can best be obtained by saturation mutagenesis in which all 19 amino acid substitutions are examined. The shortcoming of this approach is that the logistics of multiresidue saturation mutagenesis are daunting (Warren et al., 1996, Brown et al., 1996; Harrison et al., 1996; Burton and Barbas, 1994; Yelton et al., 1995; Jackson et al., 1995; Short et al., 1995; Wong et al., 1996; Hilton et al., 1996). Hundreds, and possibly even thousands, of site specific mutants must be studies. For each mutant protein, the appropriate gene construct must be made, the DNA must be transformed into a host organism, transformants need to be selected and screened for expression of the protein, the cells have to be grown to produce the protein, and finally the recombinant mutant protein must be isolated. There have been only a handful of studies where one, or at most a few, residues in an antibody have been subjected to saturation mutagenesis. Even in those studies, only some of the mutants were examined in detail (Ito et al., 1996, Chen et al., 1995, Brumell et al., 1993).

In recent years, techniques for estimating the equilibrium constant for ligand binding using minuscule amounts of protein have been developed (Parker, 1978; Blackburn et al., 1991; U.S. Pat. Nos. 5,221,605 and 5,238,808.). The inventors own work however, has shown that the ability to perform functional assays with small amounts of material can be exploited to develop highly efficient, in vitro methodologies for the saturation mutagenesis of antibodies. The inventors bypassed cloning steps by combining PCR mutagenesis with coupled in vitro transcription/translation for the high throughput generation of protein mutants. Here, the PCR products are used directly as the template for the in vitro transcription/translation of the mutant single chain antibodies. Because of the high efficiency with which all 19 amino acid substitutions can be generated and analyzed in this way, it is now possible to perform saturation mutagenesis on numerous residues of interest, a process that can be described as in vitro scanning saturation mutagenesis (Burks et al., 1997).

In vitro scanning saturation mutagenesis provides a rapid method for obtaining a large amount of structure-function information including: (i) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

The surface-displayed antibodies molecules may further be presented as fusion proteins that include reporter molecules, e.g. alkaline phosphatase, luciferase, β-lactamase, green fluorescent protein and others.

Enzyme Constructs

In addition to the fusion constructions described above, vectors for enzyme expression require that appropriate signals be provided for the synthesis of mRNA and polypeptides, and include various regulatory elements such as enhancers/promoters from both bacterial and eukaryotic systems that drive expression of the enzymes of interest in the appropriate host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

(i) Regulatory Elements

Throughout this application, the term “expression construct” is meant to include any type of genetic construct containing a nucleic acid coding for a gene product in which part or all of the nucleic acid encoding sequence is capable of being transcribed. In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

In prokaryotic gene expression, unlike eukaryotic systems the DNA is not separated from the cytoplasm by a nuclear membrane. There are also many other differences in mRNA processing of prokaryotes and eukaryotes. The first control point of prokaryotic gene expression is initiation of transcription. Transcription is initiated at positions defined precisely by promoters. Analysis of more than 100 promoters in E. coli has identified two consensus sequences positioned 350 and 10 base pairs upstream of the point in the DNA sequence where transcription begins. These sequences are involved in polymerase recognition and binding.

The topology of promoter DNA is also important, numerous bacterial promoters are known to those of skill in the art (Makrides, 1996, incorporated herein by reference). The coiling of the double helix brings the two sequence motifs into the correct position for efficient recognition and binding of RNA polymerase. In some promoters, initiation of transcription is regulated by changes in the degree of supercoiling of the DNA (Lilley and Higgins, 1991). The RNA polymerase is a multi-subunit enzyme; the core enzyme is capable of transcriptional elongation on its own but requires the addition of a further subunit (σ) in order to bind specifically to promoter sites and initiate transcription. The synthesis of σ factors in prokaryotes allows the polymerase to be directed to different sets of promoters (Helmann and Chamberlain, 1988). In B. subtilis, for example, several different σ factors are produced at different stages so that different sets of genes can be turned on at each stage.

In the absence of ancillary proteins the rate of initiation varies by up to a factor of at least a 1000 over the whole range of promoters in E. coli. The efficiency of initiation is related to the sequence and topology of the promoter region. Gene expression occurring in the absence of regulatory factors is known as constitutive. At many promoters, transcriptional initiation may be increase by binding to specific regulatory proteins.

A feature of gene organization common to prokaryotes but rare in eukaryotes is the grouping of functionally related genes into operons. In an operon, genes encoding, for example the different enzymes of a metabolic pathway are clustered and are transcribed together into a polycistronic transcript, under the control of a single promoter. This transcript is then translated to give individual proteins. Operons enable the rapid and efficient coordinate expression of a set of genes required to respond to a change in the external or internal environment of the microorganism. Premature termination plays a part in the regulation of expression of operons.

The termination of transcription occurs at specific sites. There are two types of termination events. The first (factor independent termination) occurs at sites defined by a series of U residues preceded by an inverted repeat that forms a stem-loop structure at the 3′ end of the RNA transcript. This structure interferes with the polymerase action and leads to the release of the RNA. Factor dependent termination is dependent on the interaction of protein factor rho ρ with the RNA polymerase.

In eukaryotic systems, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase promoter, the spacing between promoter elements can be increased to 50 bp apart before activity begins to decline. Depending on the promoter, it appears that individual elements can function either co-operatively or independently to activate transcription.

The particular promoter employed to control the expression of a nucleic acid sequence of interest is not believed to be important, so long as it is capable of direction the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several elements/promoters which may be employed, in the context of the present invention, to regulate the expression of the gene of interest. This list is not intended to be exhaustive of all the possible elements involved in the promotion of gene expression but, merely, to be exemplary thereof.

One may include a polyadenylation signal to effect proper polyadenylation of the gene transcript in eukaryotic cells. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

(ii) Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to ampicillin, tetracycline, neomycin, puromycin, hygromycin, DHFR, GPT, zeocin and histidinol are useful selectable markers. Alternatively, enzymes such as herpes simplex virus.

(iii) Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

Antibody and Antibody Fragment Constructs

Using the fusion technology described above, the present invention also contemplates the generation of host cells expressing, on their surface, antibodies or antibody fragments representing a library of antibodies produced in response to one or more immunogens. “Antibody” or “antibody fragment” refers to any immunologic binding agent such as IgG, IgM, IgA, IgD and IgE or any antibody-like molecule that has an antigen binding region, and includes antibody fragments such as Fab′, Fab, F(ab′)₂, single domain antibodies (DABs), Fv, scFv (single chain Fv) and the like. The techniques for preparing and using various antibody-based constructs and fragments are well known in the art.

The specificity of an antibody is determined by the complementarity determining regions (CDRs) within the light chain variable regions (V_{L}) and heavy chain variable regions (V_{H}). The F_{ab} fragment of an antibody, which is about one-third the size of a complete antibody contains the heavy and light chain variable regions, the complete light chain constant region and a portion of the heavy chain constant region. F_{ab} molecules are stable and associate well due to the contribution of the constant region sequences. However, the yield of functional F_{ab} expressed in bacterial systems is lower than that of the smaller Fᵥ fragment which contains only the variable regions of the heavy and light chains. The Fᵥ fragment is the smallest portion of an antibody that still retains a functional antigen binding site. The Fᵥ fragment has the same binding properties as the F_{ab}, however without the stability conferred by the constant regions, the two chains of the Fᵥ can dissociate relatively easily in dilute conditions.

To overcome this problem, V_{H} and V_{L} regions may be fused via a polypeptide linker (Huston et al., 1991) to stabilize the antigen binding site. This single polypeptide Fᵥ fragment is known as a single chain antibody (scFᵥ). The V_{H} and V_{L} can be arranged with either domain first. The linker joins the carboxy terminus of the first chain to the amino terminus of the second chain.

While the present invention has been illustrated with display of single chain Fᵥ molecules on the surface of the bacteria, one of skill in the art will recognize that heavy or light chain Fᵥ or F_{ab} fragments may also be used with this system. A heavy or light chain can be displayed on the surface followed by the addition of the complementary chain to the solution. The two chains are then allowed to combine on the surface of the bacteria to form a functional antibody fragment. Addition of random non-specific light or heavy chain sequences allows for the production of a combinatorial system to generate a library of diverse members.

Antibody and Antibody Fragment Gene Isolation

To accomplish construction of antibodies and antibody fragments, the encoding genes are isolated and then modified to permit cloning into the expression vector. Although methods can be used such as probing the DNA for V_{H} and V_{L} from hybridoma cDNA (Maniatis et al., 1982) or constructing a synthetic gene for V_{H} and V_{L} (Barbas et al., 1992), a convenient mode is to use template directed methods to amplify the antibody sequences. A diverse population of antibody genes can be amplified from a template sample by designing primers to the conserved sequences at the 3′ and 5′ ends of the variable region known as the framework or to the constant regions of the antibody (Iverson et al., 1989). Within the primers, restriction sites can be placed to facilitate cloning into an expression vector. By directing the primers to these conserved regions, the diversity of the antibody population is maintained to allow for the construction of diverse libraries. The specific species and class of antibody can be defined by the selection of the primer sequences as illustrated by the large number of sequences for all types of antibodies given in Kabat et al., 1987, hereby incorporated by reference.

Messenger RNA isolated from the spleen or peripheral blood of an animal can be used as the template for the amplification of an antibody library. In certain circumstances, where it is desirable to display a homogeneous population of antibody fragments on the cell surface, mRNA may be isolated from a population of monoclonal antibodies. Messenger RNA from either source can be prepared by standard methods and used directly or for the preparation of a cDNA template. Generation of mRNA for cloning antibody purposes is readily accomplished by following the well-known procedures for preparation and characterization of antibodies (see, e.g., Antibodies: A Laboratory Manual, 1988; incorporated herein by reference).

Method of Producing Monoclonal Antibodies

Generation of monoclonal antibodies (MAbs) follows generally the same procedures as those for preparing polyclonal antibodies. Briefly, a polyclonal antibody is prepared by immunizing an animal with an immunogenic composition in accordance and collecting antisera from that immunized animal. A wide range of animal species can be used for the production of antisera. Typically the animal used for production of anti-antisera is a rabbit, a mouse, a rat, a hamster, a guinea pig or a goat. Because of the relatively large blood volume of rabbits, rabbits are usually preferred for production of polyclonal antibodies.

Immunogenic compositions often vary in immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Recognized means for conjugating a polypeptide to a carrier protein are well known and include glutaraldehyde, m-maleimidobenzoyl-N-hydroxysuccinimide ester, carbodiimides and bis-diazotized benzidine.

The immunogenicity of a particular immunogen composition may be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, may also be given. The process of boosting and titering is repeated until a suitable titer is achieved. When a desired level of immunogenicity is obtained, the immunized animal can be bled and the serum isolated, stored and the spleen harvested for the isolation of mRNA from the polyclonal response or the animal can be used to generate MAbs for the isolation of mRNA from a homogeneous antibody population.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g. a small molecule hapten conjugated to a carrier, a purified or partially purified protein, polypeptide or peptide. The immunizing composition is administered in a manner effective to stimulate antibody producing cells. Rodents such as mice and rats are frequently used animals; however, the use of rabbit, sheep frog cells is also possible. The use of rats may provide certain advantages (Goding, pp. 60-61, 1986), but mice are preferred, particularly the BALB/c mouse as this is most routinely used and generally gives a higher percentage of stable fusions.

Following immunization, somatic cells with the potential for producing antibodies, specifically B lymphocytes (B cells), are selected for use in the MAb generating protocol. These cells may be obtained from biopsied spleens, tonsils or lymph nodes, or from blood samples. Spleen cells and blood cells are preferable, the former because they are a rich source of antibody-producing cells that are in the dividing plasmablast stage, and the latter because blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, 1984). For example, where the immunized animal is a mouse, one may use P3-X63/Ag8, X63-Ag8.653, NS1/1.Ag 4 I, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler & Milstein (1975; 1976), and those using polyethylene glycol (PEG), such as 37% (v/v) PEG, by Gefter et al., 1977). The use of electrically induced fusion methods is also appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unflused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. Simple and rapid assays include radioimmunoassays, enzyme immunoassays, cytotoxicity assays, plaque assays, dot immunobinding assays, and the like.

The selected hybridomas are serially diluted and cloned into individual antibody-producing cell lines from which clones can then be propagated indefinitely to provide MAbs. The cell lines may be exploited for MAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. MAbs produced by either means may be further purified, if desired, using filtration, centrifugation and various chromatographic methods such as HPLC or affinity chromatography.

Following the isolation and characterization of the desired monoclonal antibody, the mRNA can be isolated using techniques well known in the art and used as a template for amplification of the target sequence.

Amplification of Gene Fragments

A number of template dependent processes are available to amplify the target sequences present in a given template sample. One of the best known amplification methods is the polymerase chain reaction (PCR™), which is described in detail in U.S. Pat. Nos. 4,683,195, 4,683,202 and 4,800,159, and in Innis et al. (1990), each of which is incorporated herein by reference in its entirety. Briefly, in PCR™, two primer sequences are prepared which are complementary to regions on opposite complementary strands of the target sequence. An excess of deoxynucleoside triphosphates are added to a reaction mixture along with a DNA polymerase, e.g., Taq polymerase. If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction products and the process is repeated. Preferably a reverse transcriptase PCR™ amplification procedure may be performed in order to quantify the amount of target amplified. Polymerase chain reaction methodologies are well known in the art.

Another method for amplification is the ligase chain reaction (“LCR”), disclosed in EPA No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as an amplification method. In this method, a replicative sequence of RNA which has a region complementary to that of a target is added to a sample in the presence of an RNA polymerase. The polymerase will copy the replicative sequence which can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids (Walker et al., 1992).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR) involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having a 3′ and 5′ sequences of non-specific DNA and middle sequence of specific RNA is hybridized to DNA which is present in a sample. Upon hybridization, the reaction is treated with RNaseH, and the products of the probe identified as distinctive products which are released after digestion. The original template is annealed to another cycling probe and the reaction is repeated.

Other amplification methods are described in GB Application No. 2 202 328, and in PCT Application No. PCT/US89101025, each of which is incorporated herein by reference in its entirety, may be used in accordance with the present invention. In the former application, “modified” primers are used in a PCR™ like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double stranded DNA molecules are then multiply transcribed by a polymerase such as T7 or SP6. In an isothermal cyclic reaction, the RNAs are reverse transcribed into double stranded DNA, and transcribed once against with a polymerase such as T7 or SP6. The resulting products, whether truncated or complete, indicate target specific sequences.

Davey et al., EPA No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then reenter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; O'Hara et al., 1989).

Methods based on ligation of two (or more) oligonucleotides in the presence of nucleic acid having the sequence of the resulting “di-oligonucleotide”, thereby amplifying the di-oligonucleotide, may also be used in the amplification step (Wu et al., 1989).

Amplification products may be analyzed by agarose, agarose-acrylamide or polyacrylamide gel electrophoresis using standard methods (see, e.g., Maniatis et al. 1982). For example, one may use a 1% agarose gel stained with ethidium bromide and visualized under UV light. Alternatively, the amplification products may be integrally labeled with radio- or fluorometrically-labeled nucleotides. Gels can then be exposed to x-ray film or visualized under the appropriate stimulating spectra, respectively.

Semisynthetic Antibody Gene Fragments and Preparation of Mutants

Genes for antibody fragments also may be generated by semisynthetic methods known in the art (Barbas et al., 1992). Using the conserved regions of an antibody fragment as a framework, variable regions can be inserted in random combinations one or more at a time to alter the specificity of the antibody fragment and generate novel binding sites, especially in the generation of antibodies to antigens not conducive to immunization such as toxic or labile compounds. Along the same lines a known antibody sequence may be varied by introducing mutations randomly or site specifically. This may be accomplished by methods well known in the art such as mutagenesis with mismatched primers or error-prone PCR™ (Innir, 1990).

D. Host Cells, Methods of Producing Host Cells and “Dead Man” Selection

Virtually any cell may be used as a host cell, but the ease with which bacterial cell are handled makes these a preferred embodiment, at least where eukaryotic modification events (glycosylation, etc.) are not necessary. For example, mammalian cells, plant cells and yeast cells all may be employed. Preferred bacterial hosts include Gram negative bacterial cells, particularly E. coli, although Salmonella, Klebsiella, Erwinia, Pseudomonas aeruginosa, Haemophilus influenza, Rickettsia rickettsii, Neisseria gonorrhea, etc. also are suitable.

Bacterial cells in which polypeptides are expressed may be readily immobilized, thus allowing rapid recovery and efficient removal from the system. One may use membranes, dipsticks or beads through a chemically promoted coupling reaction in addition to other well-known immobilization matrices. In this manner, the cells can be separated from the solution without the need for a centrifugation step.

Bacterial cultures may be supplied in forms that have an indefinite shelf life and yet can be readily prepared for use; for example as “stab cultures” or lyophilized preparations; the user may prepare large amounts in liquid culture as needed. The reagent is thus renewable as compared with other polypeptide reagents that are “used up” and must be replaced continually. Bacterial cultures may be prepared fresh without concern about shelf-life of reagents that must be stored until use.

Selection

Growth studies of bacteria have demonstrated that the viability of positive bacteria cells (with fusion proteins) and negative cells (without fusion proteins or with truncated fusion proteins) vary significantly. The quantity of negative cells overwhelm the positive cells after the required 12 to 24 hours growth. For example, the percentage of positive cells in a library may decrease 10 to 100 fold during the growth period. Since most FACS are optimally designed for sorting mammalian cells which are 10 to 100 times larger than bacterial cell, even using the most stringent selection mode, the negative cells may be selected along with the positive cells.

Elimination of these negative cells in a presort library is a very important step for a successful sorting experiment. A number of approaches may be used to eliminate negative cells.

Negative Selection Systems

The blue-white screening system has been widely used for library screening in molecular biology. In this system, a β-galactosidase enzyme is used as a reporter protein (Maniatis, 1989), and a multiple cloning site (MCS) is located in the middle of the β-galactosidase gene. An insertion of DNA into the MCS may result in the interruption of the β-galactosidase gene. Thus, these cells with the DNA insert will not be able to produce an active β-galactosidase, and consequently cannot convert the substrate (X-gal) into a colored product, while the cells without an insert can produce β-galactosidase and hydrolyze the substrate into the indigo colored product. This allows for selection of colorless colonies which usually have an insert are picked and streaked onto a new plate. The process is repeated until a plasmid carrying a DNA insert is confirmed. Finding a positive colony with the right insert may require several rounds of selections. Since the selection is performed on an agar plate, the speed of screening is not suitable for high throughput screening (for example, millions of colonies per day).

Positive selection systems

The direct selection of an antibody with enhanced catalytic activity or altered specificity is possible when the reaction of interest can be coupled to the growth or survival of a cell, such as the release of an essential nutrient or cofactor. However, many reactions of interest do not easily lend themselves to such a selection scheme. In addition, prokaryote metabolism is very complex, and bacteria are usually capable of adapting to new metabolic pathways, resulting in a large number of false positives (Lesley, 1993).

A variety of positive selection vectors have been developed to prevent the existence of the cells carrying non-insert plasmids. All the strategies rely on the inactivation of either a lethal gene (O'Connor 1982, Henrich 1986, Kuhn 1986, Arakawa 1991, Bernard 1994), a lethal site (Hagan 1982), a dominant function conferring cell sensitivity to metabolites (Dean 1981, Burns 1984, Pierce 1992, Gossen 1992) or a repressor of an antibiotic-resistance function (Robert 1980, Nikolnikov 1984). Most of these strategies are not well adapted for general use due to their large size, the limited number of cloning sites, or the need of special host strain and special culture medium. Another problem of these systems is that they are not an expression system, and the inserted gene cannot be expressed unless it carries a complete promoter and operator sequence.

Seehaus has fused scFv DNA into the gene encoding β-lactamase (BIa) at the 3′-terminus of the signal sequence. Only those clones carrying inserts that are in frame with BIa gene can survive ampicillin selection, while others that carry out-of-frame deletions or internal stop codons are eliminated. This strategy can be applied to the Georgiou/Iverson system, however, the size of the fusion protein BIa (58 to 60 Kda) creates another problem when the strategy is implemented. Only small proteins (<53 Kda) can be displayed on the surface of E. coli and still retain significant activity (Stathopulos 1996). A Ipp-OmpA-scFv-Bla Fusion protein is too big to be displayed on the surface of bacteria. Nevertheless, this general resistance fusion strategy can be modified for our purposes.

The inventors have employed new positive (“dead man”) selection systems in the present invention using two approaches to eliminate negative cells. In the first approach, the target gene is fused with part of an antibiotic gene and used as an insert. Without the insert, the cloning vector does not process the specific antibiotic resistance, and the cells having the self-ligated vectors are eliminated by antibiotic selection. Two different plasmids were constructed with two antibiotic markers, penicillin resistance and chloramphenicol resistance, as well as the surface expression Ipp-OmpA machinery (same as pTX152 which can express the scFv on the surface of E. coli).

In one embodiment a plasmid was constructed to eliminate cells without surface-expressed scFv. A restriction site was introduced in the middle of the chloramphenicol acetyl transferase (CAT) gene and in the middle of scFv gene. The N-terminal part of CAT (200 bps) was fused to the end of scFv gene by overlapping PCR, while the cloning vector carried the C-terminal part of antibiotic gene. The cells that contain a plasmid without the appropriate insert (for example, antibody gene and N-terminal part of CAT) cannot express functional CAT protein, so they are eliminated in a chloramphenicol medium.

In another embodiment, a plasmid is constructed to eliminate cells that carry a advertition stop codon in the middle of the scFv gene. The promoter and ribosome binding site (rbs) for CAT are eliminated, and both surface expression Ipp-OmpA-scFv gene and CAT gene are under control of a single promoter and operator. Thus, the CAT gene can be transcribed but cannot be translated due to the lack of a rbs. The scFv gene and the CAT gene are fused in such a way that the stop codon of the scFv and the start codon of the CAT are arranged in the order of TAATG. When the ribosome stop at the TAA of scFV gene, it can frameshift a certain fraction of the time to the adjacent ATG codon thus restart the translation of CAT gene (Benhar). The stop codons caused by an unexpected mutation in the middle of the scFv gene will force the ribosome to fall off the mRNA early, so it will not be able to translate the CAT gene. As a result, the cells that carry the stop codon(s) in the middle of scFv gene do not have chloramphenicol resistance and are not able to survive in chloramphenicol medium.

It is envisioned that analogous cloning vectors also might improve the construction and screening of phage display libraries by reducing the number of non-insert plasmids in the presort antibody libraries, and thereby reduce the number of selection rounds required.

Of course, it is understood that other antibiotic resistance genes such as the ampicillin resistance gene and kanamycin resistance gene can be fused to the antibody gene in this system. Furthermore, any gene product that is essential for bacterial growth may be used.

Gene Transfer

Technology for introduction of DNA into cells is well-known to those of skill in the art. Four general methods for delivering a gene into cells have been described: (i) chemical methods such as calcium phosphate precipitation (Graham et al., 1973; Zatloukal et al., 1992); (ii) physical methods such as protoplast fusion, microinjection (Capecchi, 1980), electroporation (Wong et al., 1982; Fromm et al., 1985; U.S. Pat. No. 5,384,253) and the gene gun (Johnston et al., 1994; Fynan et al., 1993); (iii) viral vectors (Clapp, 1993; Lu et al., 1993; Eglitis et al., 1988a; 1988b); and (iv) receptor-mediated mechanisms (Curiel et al., 1991; 1992; Wagner et al., 1992).

E. Methods For Detecting Analytes Using Cells Displaying Scfv Antibodies

For the first time, a competitive immunoassay has been developed that takes advantage of anti-analyte binding antibodies immobilized on a bacterial cell surface. Several advantages of the disclosed immunoassay include generally the convenience, wide applicability and the simplicity, rapidity and sensitivity of the assay. In addition, selected host cells displaying an polypeptide of interest may be used to stimulate an immune response.

The first step in the development of whole cells suitable for the detection of analytes is the screening of antibody libraries displayed on the cell surface. Cells displaying antibodies having affinity for a desired analyte are isolated. First, a library of cell surface displayed proteins is prepared as described elsewhere in the specification. For example a library of surface displayed scFv antibodies can be prepared as described in Example 7. Once an expression library has been prepared, the selected antigen for which one desires to identify and isolate specific antibody or antibodies is labeled with a detectable label. There are many types of detectable labels, including fluorescent labels, the latter being preferred in that they are easily handled, inexpensive and non-toxic. The labeled antigen is contacted with the cells displaying the antibody expression library under conditions that allow specific antigen-antibody binding. Conditions can be varied so that only very tightly binding interactions occur; for example, by using very low concentrations of labeled antigen.

Identifying the antibody or antibody fragment expressing cells may be accomplished by methods that depend on detecting the presence of the bound detectable label. A particularly preferred method for identification and isolation is cell sorting or flow cytometry. One aspect of this method is fluorescence activated cell sorting (FACS).

Following selection of high affinity clones, the production of soluble antibodies can be achieved easily without the need for further subcloning steps. Thus, the clones may be maintained under standard culture conditions and employed to produce the selected antibody. Production of antibody is limited only to the scaleup of the cultures.

The invention further includes competitive binding assays using cells with antibodies or analyte-combining antibody fragments expressed on the outer cell surface. As used in the context of the present invention, analyte is defined as a species that interacts with a non-identical molecule to form a tightly bound, stable complex. For practical purposes, the binding affinity is usually greater than about 10⁶ M⁻¹ and is preferably in the range of 10⁹-10¹⁵ M⁻¹. The analyte may be any of several types of organic molecules, including alicyclic hydrocarbons, polynuclear aromatics, halogenated compounds, benzenoids, polynuclear hydrocarbons, nitrogen heterocyclics, sulfur heterocyclics, oxygen heterocyclics, and alkane, alkene alkyne hydrocarbons, etc. Biological molecules are of particular interest, including amino acids, peptides, proteins, lipids, saccharides, nucleic acids and combinations thereof. Of course it will be understood that these are by way of example only and that the disclosed immunoassay methods are applicable to detecting an extraordinarily wide range of compounds, so long as one can obtain an antibody that binds with the analyte of interest.

The disclosed whole cell immunoassay methods allow rapid detection of a wide range of analytes and are particularly useful for determination of polypeptides. The methods have been developed to take advantage of the binding characteristics of bacterial cell surface exposed anti-analyte antibodies. Such surface displayed antibodies are stable and bind readily with specific analytes. This unique form of protein expression and immobilization thus has provided the basis of an extremely rapid competitive assay that may be performed in a single reaction vessel in an “add and measure” format. Such assays can be described as “one-pot” reactions that make possible in situ detection of an analyte.

A particular advantage of cell surface expressed antigen-binding antibodies is that the antibody is attached to the outer membrane of the cell. The cells therefore act as a solid support during the assay, thereby eliminating many of the manipulations typically required in preparing reagents required for existing immunoassay techniques. Optionally, cells with the antibody displayed on the surface may themselves be attached to a solid support such as a membrane, dipstick or beads to further facilitate removal of the cells following the assay.

The immunoassays of the present invention may be used to quantitate a wide range of analytes. Generally, one first obtains the appropriate host cell culture where the anti-analyte antibody is displayed on the host cell surface, calibrates with standard samples of analyte, then runs the assay with a measured volume of unknown concentration of analyte.

In conducting a competitive immunoassay in accordance with the disclosed methods, one first obtains a host cell that expresses an analyte binding antibody. The host cell is then contacted with a standard analyte sample that contains a known amount of an analyte linked to a detectable label employing conditions effective for forming an immune complex. Once calibration is completed, the same procedure is used with a second host cell that has the same antibody or analyte-combining fragment expressed on its surface, except that in addition to the standard labeled analyte sample, a test sample in which an unknown amount of analyte is to be determined is added. One is not limited to using the same host cell in this procedure.

In the actual assay, a known amount of the antibody-covered cells are placed in a solution of a known concentration of the analyte-conjugate along with an unknown concentration of the analyte (the test solution). The analyte conjugate competes with free analyte in solution for binding to the antibody molecules on the cell surface. The higher the concentration of analyte conjugate in the solution, the fewer molecules of fluorescein analyte conjugate bind on the surface of the cells, and vice versa.

The mixture is centrifuged to pellet the cells, and the fluorescence of the supernatant is measured. The assay is quantitative because the amount of observed fluorescence is proportional to the concentration of analyte in the test sample, i.e., if there is a very low concentration of analyte to compete with the fluorescein conjugate, then most of the conjugate will bind to the cells and will be removed from solution. The more molecules of analyte in solution, the more molecules of analyte bind to the antibodies thereby preventing the conjugate from binding. In this case, more fluorescein conjugate remains in the supematant to give a stronger fluorescence signal. The assay can be calibrated to generate a quantitative measurement of the unknown concentration of analyte. The entire assay requires less than one hour. Fluorescence determinations may be made with a basic fluorimeter.

The present invention involves a novel method of carrying out competitive immunoassays using antibodies attached to the surface of cells. The disclosed immunoassays are useful for binding, purifying, removing, quantifying or otherwise generally detecting analytes. Antibodies expressed on bacterial cell surfaces have been shown surprisingly adaptable for use in competitive immunoassay procedures. In a particular example, the analyte digoxin, a cardiac glycoside, was determined using fluorescein-digoxin conjugate. The assay was quantitative with a sensitivity in the nanomolar range.

Cells expressing an antibody fragment on their surface may also be linked to a solid support, such as in the form of beads, membrane or a column matrix, and the sample suspected of containing the unwanted antigenic component applied to the immobilized antibody. A purged or purified sample is then obtained free from the unwanted antigen simply by collecting the sample from the column and leaving the antigen immunocomplexed to the immobilized antibody.

Detection of immunocomplex formation is well known in the art and may be achieved through the application of numerous approaches. These approaches are typically based upon the detection of a label or marker, such as any of the radioactive, fluorescent, chemiluminescent, electrochemiluminescent, biological or enzymatic tags or labels known in the art. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotinlavidin ligand binding arrangement, as is known in the art.

The first added component that becomes bound within the primary immune complexes may be detected by means of a second binding ligand that has binding affinity for the surface expressed antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the surface expressed antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The competitive immunoassay discussed above requires one to set up a control sample. This sample comprises a second host cell expressing an analyte binding antibody. This is contacted with a known amount of analyte linked to a detectable label and a known amount of unlabeled analyte, i.e. the analyte to be detected or determined in the assay. After formation of the immunocomplexes, one measures the free label in solution after the cells have been separated. This measures the amount of residual detectable label as the decrease in, e.g., fluorescence emission, from which the amount of unknown analyte may be determined. A preferred fluorescent label is fluorescein. The inventors have found that measurement of the amount of residual label that is not bound to antibodies is proportional to the amount of analyte label in solution. This provides the basis for quantitative measure in that the increase in the amount of label is directly proportional to the analyte. Alternatively, the label may be measured in the complexes. In this type of measurement, the analyte is inversely proportional to the amount of label.

Of course fluorescence labeling, while preferred, does not preclude the use of other detectable agents such as chemiluminescent agents, electrochemiluminescent agents, radioactive labels, enzymatic labels that form a colored product with a chromogenic substrate as well as other fluorescent compounds. A preferred fluorescent label is fluorescein while Ru(bpy)₃²⁺ is preferred for use as an electrochemiluminescent agent.

The invention is readily adaptable to the determination of multiple analytes. This is achieved using two or more different analyte-binding antibodies expressed in separate host cells. It is also possible to surface express more than one antibody on the surface of a particular host cell; however, this may cause interference in binding. One will, in these situations, use different detecting agents; for example, two different fluorescent labels, each with distinct emissions, such as fluorescein which emits at 520 nm and Texas Red which emits at 620 nm.

In certain uses of the assay, cells containing antibodies on the surface are produced as described previously. Antibodies known to bind tightly and specifically to a molecule of interest are employed. The molecule could be a medically relevant molecule, a marker molecule used in a scientific study, a pesticide of environmental concern in groundwater, etc. A covalent conjugate of the molecule with a fluorescent moiety such as fluorescein is synthesized for use as a probe in binding. Other detection agents include radioactive compounds, enzyme conjugates, chemiluminescent reagents such as luciferase and electrochemiluminescent reagents such as Ru(bpy)₃²⁺. (Yang et al., 1994; Blackburn et al., 1991). The assay may also be carried out using tritium as the labeling agent for the antigen and performing a radioimmunoassay. The radioactivity may be detected using a scintillation counter to measure binding constants up to 10⁻⁸ or 10⁻⁹ M⁻¹.

To perform the preferred assay, a known amount of the antibody-covered cells are placed in a solution of a known concentration of the molecule-fluorescein conjugate along with an unknown concentration of the molecule. The molecule-fluorescein conjugate competes with free molecules in solution for binding to the antibody on the cell surface. The higher the concentration of the molecule in the solution, the fewer molecules of fluorescein-molecule conjugate will bind to the surface of the cells, while lower concentrations of the molecule will result in more of the conjugate bound to the cell surface.

Before measuring residual fluorescence, the cells are removed from the solution, most conveniently by pelleting, and the fluorescence of the supernatant measured. The assay is quantitative because the amount of observed fluorescence is proportional to the concentration of the molecule in the unknown sample. If there is a very low concentration of the molecule to compete with the fluorescein conjugate, most of the conjugate will bind to the cells and will be removed from the solution. The higher the concentration of the molecule in solution, the more molecules bind to the antibodies thereby preventing the conjugate from binding. In this case, more fluorescein conjugate remains in the supernatant to give a stronger fluorescent signal. The assay can be calibrated to generate a quantitative measurement of the unknown concentration of the molecule. The entire assay takes less than an hour and requires only a basic fluorimeter.

As part of the invention, immunoassay kits are also envisioned comprising a container having suitably aliquoted reagents for performing the foregoing methods. For example, the containers may include one or more bacterial cells with particular surface expressed analyte-binding antibodies. Suitable containers might be vials made of plastic or glass, various tubes such as test tubes, metal cylinders, ceramic cups or the like. Containers may be prepared with a wide range of suitable aliquots, depending on applications and on the scale of the preparation. Generally, this will be an amount in conveniently handled form, such as freeze-dried preparations, and sufficient to allow rapid growth of the bacterial cells as required.

Such kits may optionally include surface-expressed antibodies in host cells that are immobilized on surfaces appropriate for the intended use. One may for example, provide the cells attached to the surface of microtiter plates, adsorbent resins, cellulose (e.g. filter paper), polymers, glass beads, etc.

F. Methods for Screening Libraries of Surface Displayed Polypeptides for the Isolation of Polypeptides Capable of Binding to Desired Target Molecules

The present invention discloses general methods for the screening of polypeptide libraries for the isolation of polypeptides that recognize and binding to desired target molecules with high affinity. Cell expressing such high affinity polypeptides can then be readily employed for immunoassays as described in section E. of the specification.

The screening of combinatorial libraries of polypeptides is greatly facilitated by the display of the polypeptides on the surface of host cells. Simply, a cell population where each cell displays a different polypeptide is contacted with a desired ligand. The ligand is labeled such that it can allow the facile separation of cells that display polypeptides capable of binding the ligand. Examples of labels suitable for the purposes of this invention include fluorescent dyes and magnetic particles. Alternatively the desired target molecule can be immobilized on a suitable solid support. Cells producing surface displayed polypeptides capable of binding the desired target molecule thus adhere to the immobilized support and can be readily separated from cells that do not bind to the immobilized support.

FACS Separation of Desired Cells Libraries of surface-displayed polypeptides are rapidly and efficiently sorted using fluorescence activated cell sorting techniques (FACS). FACS permits the separation of subpopulations of cells initially on the basis of their light scatter properties as they pass through a laser beam. Since cells are tagged with fluorescent-labeled product, they can are characterized by fluorescence intensity and positive and negative windows set on the FACS to collect label⁺ (bright fluorescence) and label⁻ (low fluorescence) cells. Positive and negative windows are set to collect label⁺ and label⁻ cells, respectively. Cells are sorted at a flow rate of about 3000 cells per second and collected in positive and negative cells.

Identification of antibody-expressing bacteria by FACS is directly based on the affinity for the soluble hapten thus eliminating artifacts due to binding on solid surfaces. This means only the high affinity antibodies are recovered by sorting following binding of low concentrations of fluorescently labeled antigen. There is no analogous method for specifically selecting phage with very high affinity. Additionally, the sorting of positive clones is essentially quantitative. It is limited only by the accuracy of the flow cytometer, which is on the order of 95%. In contrast with phage technology, the efficiency of selection is not limited by avidity effects because screening does not depend on binding to a surface having multiple antigens and thus the potential for multivalent attachment sites.

Magnetic Separation of Desired Cells E. coli cells with surface expressed polypeptides can be incubated with paramagnetic particles (e.g. Miltenyi Biotec, Bergisch Gladbach) that themselves are coated with an antigen of interest (Radbuch et al. 1994). Paramagnetic particles are available with a variety of surface derivatization chemistries to allow for the covalent attachment of a wide range of antigens. Bacteria having polypeptides with high affinity for the antigen remain bound to the magnetic particles, and the complexes isolated following washing steps in a strong magnetic field that retains the paramagnetic beads. Alternatively, cells labeled with paramagnetic particles can be separated in a continuous magnetic separator.

Separation of Cells by Adsorption onto Supports with Immobilized Target Molecules In another embodiment of the present invention, cells displaying polypeptides that bind to the desired target molecule may be isolated via selective adsorption onto solid matrices. In this case the cell population displaying the polypeptide library is contacted with a solid support in which the antigen is covalently immobilized via standard chemical immobilization methodologies. Cells that display polypeptides capable of interacting with the immobilized target molecules are retained on the solid support and can be separated from non-binding cells. Following several washes with buffer to remove non-specifically adsorbed cells, the cells that are bound via specific interactions are employed for further studies. Such specifically-bound cells can be dissociated from the solid support either by adding large concentrations of the soluble desired molecule to serve as a competitor or, alternatively by adding growth media to allow the cells to grow. In the latter case the progeny of the bound cells is released from the solid support.

G. Methods for the Selection of Evolved Enzymes

The immobilization onto micron sized particles generally involves bacteria being entrapped within agarose gel microdroplets (AGMs). This method involves entrapping microorganisms in AGMs (10 to 100 microns in diameter) which are surrounded by a hydrophobic (low dielectric) fluid, subsequently distinguishing occupied and unoccupied AGMs with colorimetric or fluorescence indicators, counting both occupied and unoccupied AGMs and applying statistical analyses to arrive at enumeration. It is possible to use a single preparation of AGMs containing a range of AGM sizes, to simultaneously provide a viable enumeration of growing and non-growing cells.

The microdroplets are produced by first emulsifying a solution containing bacteria and melted agarose. This results in the formation of liquid microdroplets suspended in oil. By lowering the temperature at which the agarose is made to polymerize, thus forming micron sized droplets containing bacterial cells. AGMs of approximately 10 μM in diameter have been made using standard procedure. The conditions for making AGMs that are occupied by one cell are well known to those of skill in the art (Weaver et al., 1991).

This technique starts with a conventional cell suspension and generates a large number (10⁶) of AGMs/ml by adding the cells to molten agarose and dispersing into mineral oil. This suspension of AGMs is then transiently cooled to a gelation state (Weaver et al., 1984; Weaver, 1986; Williams et al., 1987; Weaver et al., 1988). Poisson statistics allow the measurement of size of those AGMs that have a high probability of containing zero or one initial cell or colony forming units. The AGMs can be transferred out of the mineral oil into a suitable growth medium and incubated to allow for the formation of microcolonies. These can then be stained with fluorescent dyes for one or more generic indicators of biomass, for example, nucleic acids (stained with propidium iodide) or proteins (stained with FITC) and then measured using flow cytometry generally with single cell resolution. DNA staining may also be used and are well known to those of skill in the art (Bliss, 1990; Powell, 1989).

This assay method is applicable to any cell type which is amenable to being cultured in a gel-like matrix. The assay method has been successfully demonstrated on mammalian, fungal and bacterial cells (Weaver et al., 1991). The method is sensitive to subpopulations with different growth rates and may be used for working with a mixed population of cells without the need for strain specific stains and is applicable to any growth based assay.

For growth based assays the use of AGMs for the isolation of individual cells within AGMs thereby allowing both monoculture and mixed cell populations to be assayed. Further AGMs provide confinement of progeny of individual cells within AGMs, rapid measurement of large numbers of individual microcolonies by flow cytometry and a hybrid of plating and cell suspension culture. Other fundamental advantages include the extreme permeability of AGMs allowing convenient and rapid changes in culture conditions. AGMs respond rapidly to changes in physical conditions (heat, electric field, light ionizing radiation), and they are small and robust enough to be handled in a manner similar to the handling of cells i.e. suspension, pipetting centrifuging and the like. It is possible to add additional matrix components (separately or in combination with agarose) to approximate a natural, complex extracellular environment and support the growth of cells which do not grow in agarose alone (Scott, 1987; Nilsson et al., 1987; Akporiaye et al., 1988)

A variety of substrates can be used to assay the activity of, and ultimately select for, desirable mutant enzymes out of surfaceexpressed mutant enzyme libraries. Here the gene for an enzyme to be mutated is expressed on the surface of bacteria such as E. coli by cloning the gene for the enzyme into a surface-expression vehicle such as the LPP-OmpA system (Francisco et al., 1992, 1993). Mutant enzyme libraries can be created from these gene constructs using known methods such as chemical mutagenesis, error-prone PCR or amplification in a mutator strain. Identification of mutant enzymes with desirable properties such as novel substrate selectivity or remarkable catalytic activity can be achieved using substrates that change an assayable property, i.e. fluorescence intensity, ratio of multiple fluorophore emissions, antibody detectable structural changes etc., upon catalytic action of the enzyme.

Numerous formats can be used to create the substrates with assayable properties. For example, when assaying hydrolytic enzymes (lipases, esterases, phosphatases, etc.), a substrate can be synthesized that has a fluorophore (fluorescein, bodipy, etc.) and a quencher (eosin, tetramethyl rhodamine, etc.) attached on either side of the hydrolyzed bond. Enzymatic cleavage will result in separation of the fluorophore from the quencher leading to an assayable increase in fluorophore fluorescence.

For an enzyme that synthesizes a single product from two or more substrates (ligases, aldolases, kinases, various biosynthetic enzymes), different fluorophores or chromophores can be attached to the different individual substrates. The ratio of the emissions can be determined, and a one-to-one ratio would indicate enzymatic reaction. Alternatively, only one substrate could contain the fluorophore, and the presence of fluorophore emission on a product that is preferentially retained (see below) would indicate enzymatic reaction. Additional formats could be envisioned in which quenchers are attached to one of the substrates and a fluorophore is attached to another, so that reaction leads to quenching of emission.

Finally, antibodies could be created that bind only products, not reactants, analogous to the detection used for cat-ELISA experiments (Tawfik et al. (1993)). Here the product-specific antibodies could be labeled with a fluorophore. The retention of the fluorophore emission would indicate the presence of product and hence an enzymatic reaction. This antibody labeling method should be applicable to almost any enzymatic process, regardless of reaction type, as long an antibodies could be found that can discriminate between substrates and products.

Since the mutant enzymes will be displayed on the surface of the bacteria, there is no impediment to diffusion of the assayable substrate to the mutant enzymes. This direct substrate access will allow substrates of virtually any size or shape to be used to assay for catalytic activity, including polymeric species such as nucleic acids or peptides, that would not reliably diffuse into cells.

An essential element of the experimental design is that the spectroscopically identifiable product must remain associated with the bacterium having the surface-expressed enzyme. This can be accomplished by either of two methods.

In the first method, the product is electrostatically trapped on the bacterial surface. Bacteria such as E. coli have negatively charged surfaces so that molecules with positive overall charge are retained on the cellular surface. Substrate systems in which the product(s) of the enzymatic reactions posses substantially more positive charge than substrate(s) will have product preferentially retained on the bacterium harboring the surface expressed mutant enzyme that carried out the reaction. By using low salt media, even after washing steps, the product can be retained on the bacteria long enough for screening and selection of the bacterial library using FACS. Here the gate of the FACS could be set to select for any number of parameters such as the presence of an unquenched fluorescent signal, the proper ratio between two fluorophores of different emission wavelengths, the specific retention of a single fluorophore, or the presence of product specific antibodies with covalently attached fluorophores. One or several rounds of mutagenesis and/or FACS selection could be used for enrichment of bacteria expressing desired mutants, and the selected bacteria can be plated directly for screening of individual colonies, or regrown in liquid media in preparation for further rounds of FACS selection.

In the second method, the enzymatic reaction with substrate is carried out in AGM's (Weaver et al., 1991) with enclosed bacteria from the surface-expressed enzyme library. A solvent is chosen, such as mineral oil, to suspend the AGM's such that the product of the enzyme reaction is only soluble in the aqueous environment of the AGM surrounding the cell, not the mineral oil. This will ensure that the AGM with the most active enzyme will accumulate the most product. If the product fluorescence can be distinguished from substrate, i.e. via unquenching (hydrolysis of a fluorophore/quencher substrate) or quenching (synthesis of a product with both a fluorophore and a quencher from substrates that contained only one or the other) then the AGM's with desired enzyme activities could be isolated by FACS or via fluorescence microscopy using a micromanipulator.

For any of the previously mentioned selection strategies, the substrates can be changed dramatically all at once, or incrementally over the course of several rounds of selection. Either way, enzymes with dramatically different activities compared to wild type will be isolated.

I. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Surface Expression of Anti-digoxin Single Chain Fv Antibodies

E. coli strain JM109 [endA1 recAI gyrA thi-1 hsdR17 (rₖ−, mₖ+) relA1 supE44 Δ(lac-proAB)/F′ traD36proAB lacI^{q} lacZΔM15] was used for all studies. pTX101 codes for an Lpp-OmpA-β-lactamase fusion (Francisco et al., 1992). pTX152 codes for an Lpp-OmpA-scFᵥ(digoxin) fusion, where the scFᵥ(digoxin) is an anti-digoxin single chain Fᵥ consisting of the heavy- and light-chain variable regions (V_{H} and V_{L}). The V_{H} and V_{L}, joined by a 15 amino acid [(Gly)₄Ser]₃ linker (Huston et al., 1988), were amplified from messenger RNA isolated from two separate anti-digoxin hybridomas. An 11 amino acid peptide from the Herpes Simplex Virus glycoprotein (Novagen) was introduced at the C-terminus of the scFᵥ for analytical purposes. The presence of the HSV peptide allowed detection of the scFᵥ(digoxin) protein by reaction with a monoclonal antibody specific for the 11 amino acid epitope. The sequence of the single chain Fᵥ antibody fragment is disclosed in SEQ ID NO: 2. pTX152 was constructed by first removing the bla from pTX101 by digestion with EcoRI and BamHI. The amplified gene coding for the anti-digoxin scFᵥ was then digested with EcoRI and BamHI and ligated into pTX101. Both pTX101 and pTX152 carried the chloramphenicol resistance gene. Cultures were grown in LB medium (Dilfco) supplemented with 0.2% glucose and chloramphenicol (50 μg/ml) at a temperature of either 24° C. or 37° C.

Overnight cultures grown at 24° C. were harvested, resuspended in PBS at OD₆₀₀=2.0 and lysed by passage through a French pressure cell at 20,000 psi. The lysates were then diluted with 1 volume of phosphate buffered saline containing 2.0% bovine serum albumin (PBS/2% BSA) and 5 mM of the protease inhibitor phenylmethylsulfonyl fluoride (PMSF). 96 well microtiter plates were incubated overnight at 37° C. with 100 μl of 100 μg/ml of either bovine serum albumin (BAS) or digoxin-conjugated BSA (digoxin-BSA) in 0.1 M sodium carbonate buffer (pH 9.2). All subsequent steps were carried out at room temperature. The wells were fixed for 5 min with 100 μl methanol and were then blocked for 45 min with 200 μl of pBS/1% BSA. After removing the blocking solution, the wells were incubated for 2 hr with 100 μl of lysates, washed 3 times with 200 μl PBS/0.1% Tween 20 and incubated for 1 hr with 100 μl/well of monoclonal antibodies against the HSV peptide or antiserum against β-lactamase. The wells were again washed 3 times with PBS/0.1% Tween, incubated for 1 hr with 100 μl of the appropriate secondary antibodies conjugated with horseradish peroxidase and were finally washed 5 times with PBS/0.1% Tween and 2 times with PBS. After addition of the substrate 2,2′-azino-bis(3-ethylbenzthiazoline-6-sulfonic acid) (Pierce, Rockford, Ill.) the absorbance of each well was measured at 410 nm.

Whole cell ELISAs were performed as described above except that 100 μl samples of overnight cultures that had been resuspended in PBS/1% BSA at OD₆₀₀=1.0 were used instead of cell lysates.

For fluorescence microscopy, overnight cultures grown at 24° C. were harvested, resuspended at OD₆₀₀=0.5 in PBS containing 10⁻⁷ M fluorescein-conjugated digoxin (digoxin-FITC; ILS LTD, London) and were incubated at room temperature for 1 hr. Prior to microscopy the cells were washed once with PBS and resuspended in equal volumes of PBS and Vectashield mounting medium (Vector Laboratories) at an OD₆₀₀ of approximately 2.0.

The protein composition of whole cell membrane fractions isolated from overnight cultures was analyzed by SDS-PAGE on 12% acrylamide gels and by Western blotting using anti-HSV monoclonal antibodies obtained from Novagen Inc. (Madison, Wis.), and anti-OmpA antiserum. Whole membrane fractions were prepared as described in Francisco et al. (1992).

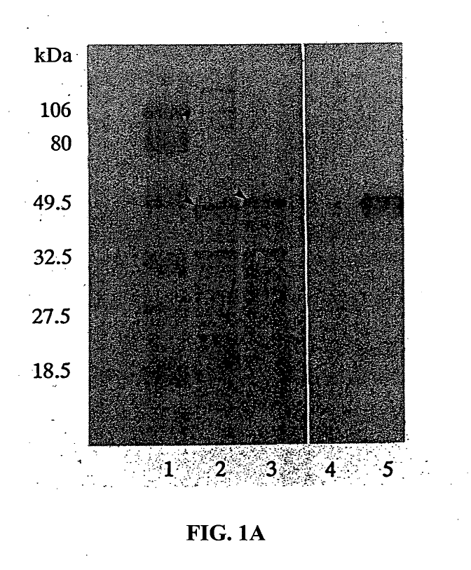

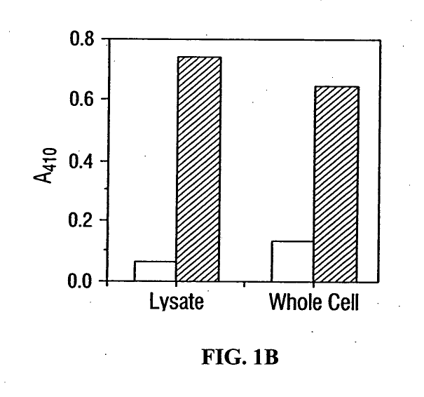

Cells were grown in liquid culture at 37° C. and used to isolate total membranes. The presence of a band of the expected size (42 kDa) was detected in Western blots of whole cell membranes probed with antibodies specific for OmpA and for the HSV peptide (FIG. 1A). Cells containing pTX152 produced a protein which reacted with both the HSV-specific and the OmpA specific sera, as expected for the Lpp-OmpA-scFᵥ(digoxin) fusion. Control cells containing pTX101 reacted only with OmpA antiserum. The lower molecular weight band in lanes 1 and 2 corresponds to the intact OmpA protein of E. coli.

The near absence of lower molecular weight bands crossreacting with either the anti-HSV or the anti-OmpA antibodies indicated that the scFᵥ(digoxin) was not subjected to proteolysis ostensibly because it was anchored on the cell surface and consequently is physically separated from intracellular proteases. The intensity of the Lpp-OmpA(46-159)-scFᵥ(digoxin) band in FIG. 1A is comparable to that of the native OmpA band. The latter is a highly expressed protein that is present in the E. coli outer membrane at about 100,000 copies per cell (Lugtenberg & Van Alphen, 1983). The level of expression Lpp-OmpA(46-159)-scFᵥ(digoxin) is on the order of 50,000-100,000 copies per cell.

The scFᵥ domain of the Lpp-OmpA-scFᵥ(digoxin) fusion protein encoded by pTX152 was shown by ELISA to bind specifically to the hapten digoxin. Whole cell lysates from JM109/pTX152 and from JM109/pTX101 as control were incubated on microtiter wells that had been coated with either digoxin-conjugated BSA (digoxin-BSA) or unconjugated BSA. Subsequently, the wells were treated with antibodies against the HSV peptide or β-lactamase to detect Lpp-OmpA(46-159)-scFᵥ(digoxin) or Lpp-OmpA(46-159)-β-lactamase, respectively. FIG. 1A shows that lysates from JM198/pTX152 bound specifically to wells coated with digoxin-BSA but not to unconjugated BSA, whereas the lysates from the control strain, JM109/pTX101, did not give a signal with either. Thus, Lpp-OmpA(46-159)-scFᵥ(digoxin) is active and can bind to the hapten specifically.

FIG. 1B shows the results of ELISAs using intact cells. Samples containing the same number of cells were used in all the studies. Cells containing the control plasmid, pTX101, gave the same low signal when incubated on microtiter wells coated with either unconjugated BSA or with digoxin-BSA. A similar weak signal was detected with JM109/pTX152 incubated on BSA-coated wells and is presumably due to non-specific binding. In contrast, a much higher absorbance was evident in wells coated with the digoxin-BSA conjugate indicating that there are active fusion protein molecules on the cell surface.

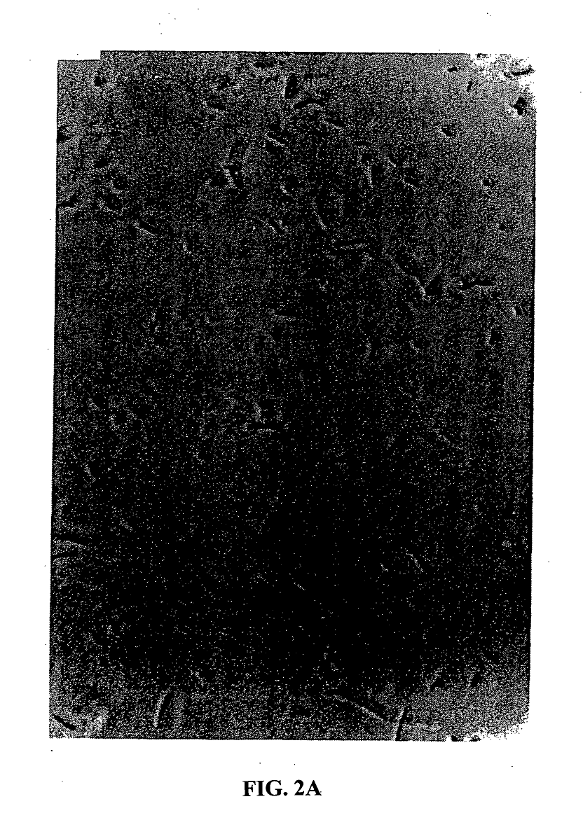

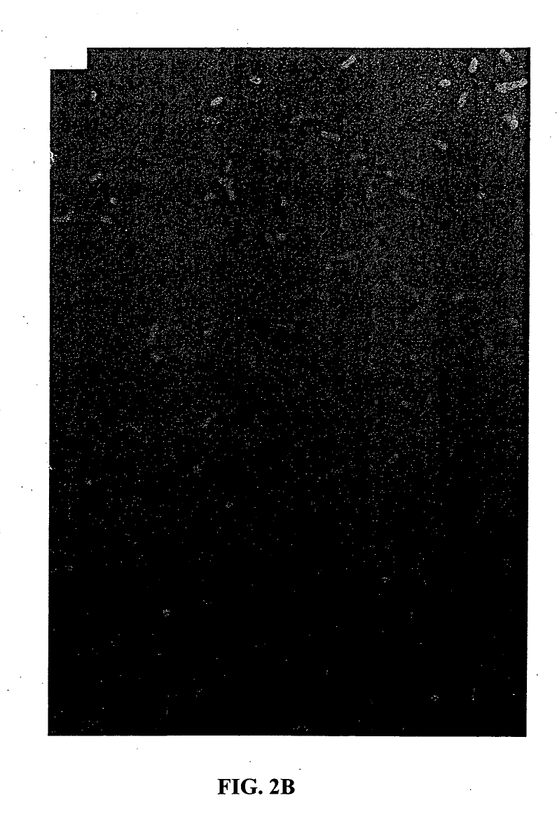

The display of the active scFᵥ antibody on the cell surface was confirmed by fluorescence microscopy (FIG. 2B). JM109/pTX152 cells were grown overnight at 24° C., incubated with a 1×10⁻⁷ M solution of a digoxin-FITC conjugate for 1 hour and washed. As shown in FIG. 2A and FIG. 2B, all of the cells visible with phase contrast microscopy gave a strong fluorescence signal. In control studies, when JM109/pTX101 cells were incubated with the same concentration of digoxin-FITC and then washed, none of the cells became fluorescently labeled. Furthermore, protease treatment drastically reduced the ability of the cells to bind fluorescein-digoxin judging from the generation of a signal detectable by FACS.

The intensity of the fluorescence signal from JM109/pTX152 was dependent on the cell growth temperature and was much higher for cultures grown at 24° C. instead of 37° C. This was consistent with previous results showing that the amount of proteins expressed on the surface of E. coli by fusion to Lpp-OmpA(46-159) increased as the temperature is decreased (Francisco et al., 1992; 1993). Assuming that the efficiency of surface display in this case is similar to that of β-lactamase (Francisco et al., 1992), then at 24° C. virtually all the scFᵥ antibody chains must be accessible on the cell surface.

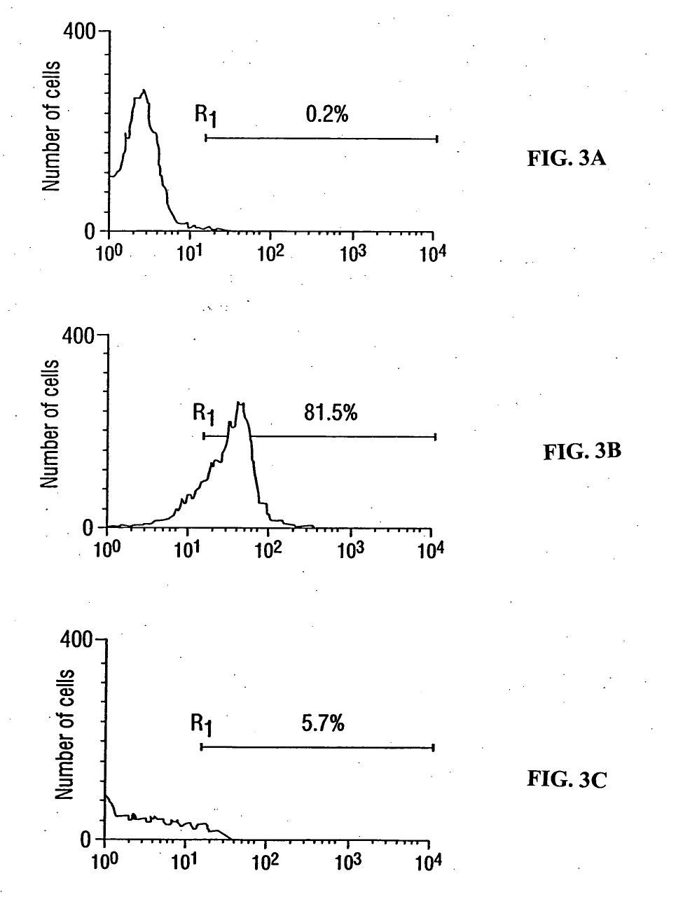

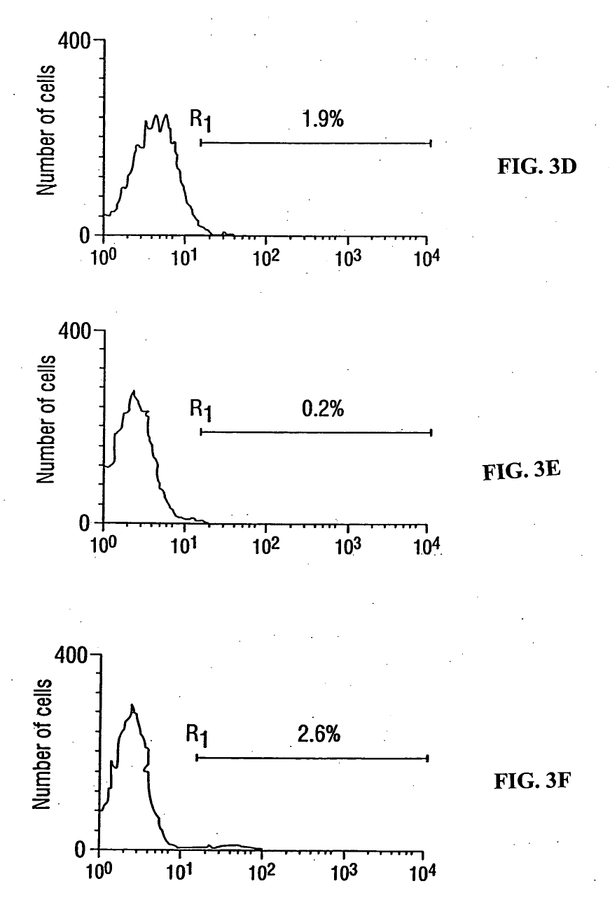

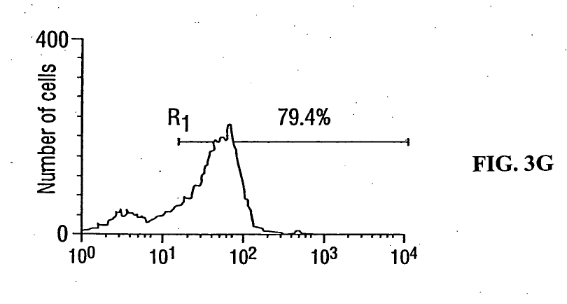

Samples of 10⁸ cells per ml from cultures grown at 24° C. were incubated with digoxin-FITC at 10⁻⁷ M, washed in buffer and diluted to 3×10⁶ cells per ml prior to sorting. The samples were then analyzed using a FACSort flow cytometer. FIG. 3A and FIG. 3B show that the fluorescence intensity of JM101/pTX152 cells expressing a surface displayed recombinant antibody specific for digoxin was substantially higher than the intrinsic background signal of control E. coli cells. JM101/pTX101 expressing Lpp-OmpA(46-159)-β-lactamase is used as a control.

When JM109/pTX152 cells were preincubated with an excess of free digoxin prior to incubation with the digoxin-FITC conjugate, the fluorescence intensity of the cells was the same as for the background (FIG. 3D). This specific inhibition was also seen using fluorescence microscopy, and demonstrates that the surface expressed scFᵥ(digoxin) specifically binds the fluorescently labeled hapten in the binding site and is not the result of nonspecific interactions.

Treatment of intact cells with trypsin prior to incubation with digoxin-FITC almost completely eliminated the population of fluorescently labeled cells detected by flow cytometry (FIG. 3C). In gram-negative bacteria, the outer membrane serves as a barrier to preclude the diffusion of large extracellular molecules such as proteins. The action of trypsin is limited to the proteolysis of proteins exposed on the external surface of E. coli (Kornacker & Pugsley, 1990). Thus, the above result provides further evidence that the active scFᵥ(digoxin) is indeed accessible on the outer surface, free to interact with molecules in solution.

The scFᵥ(digoxin) binding sites appeared to be fully saturated at concentrations of digoxin-FITC above 10⁻⁷ M. Appreciable fluorescent signal was clearly detected even at digoxin-FITC concentrations of 10⁻⁹ M. These results are consistent with a binding constant that is at least within an order of magnitude of the reported affinity of a soluble anti-digoxin scFᵥ antibody (Huston et al., 1988).

### Example 2

### Enrichment of Cells Displaying scFᵥ Antibodies by FACS

Antibody expressing cells were sorted essentially quantitatively from an excess of control E. coli in a single step. Specifically, in mixtures containing JM109/pTX101 control cells at an excess of either 100:1 or 1,000:1, the fraction of the total population that was sorted in the high fluorescence intensity window was 1.1% and 0.1% respectively (after subtracting the background), as expected from the ratio of input cells.

The use of FACS for isolating rare clones from a very large excess of background was also demonstrated. JM109/pTX101 (cells displaying an unrelated protein on the cell surface) and JM101/pTX152 (cells displaying the scFv(digoxin) antibodies) were mixed at a ratio of 100,000:1 and labeled with digoxin-FITC. Following washing to remove any non-specific binding of the digoxin-fluorescein conjugate on the control E. coli, 500,000 cells were run through the FACSort flow cytometer. A wide sorting gate, i.e., the minimum fluorescence required for acceptance of an individual cell, was selected such that up to 0.2% of the control cells fell within the sorting window. This ensured that all the scFᵥ(digoxin) expressing cells would be recovered. The cells having an allowable fluorescence signal were collected and grown in fresh media at 37° C.

An aliquot from that culture was used to inoculate fresh media and incubated at 24° C. Cells grown at this lower temperature were used for FACS because of the higher extent of surface display of the scFᵥ(digoxin) antibody on the surface of cells grown at 24° C. 500,000 cells from this culture were run again through the FACSort and those with a fluorescence within the allowable window were collected automatically under sterile conditions. The sorted population was grown first at 37° C. and then subcultures at 24° C. and was subjected to a final round of sorting as above.

To ensure the complete absence of artifacts due to non-specific cell adhesion in the flow path of the FACSort, each run was followed by extensive washing with bleach. FIG. 3E, FIG. 3F and FIG. 3G show the cell fluorescence distribution for the sorting runs. After only two rounds of growth and sorting, the fluorescence intensity of 79% of the cell population fell within the positive window. A similar enrichment was reproducibly obtained in three independent studies. These results were not due to a growth advantage of the cells expressing Lpp-OmpA(46-159)-scFᵥ(digoxin), since successive regrowth of the input cell mixture in the absence of sorting did not result in any detectable enrichment.

To verify that the cells with the increased fluorescent signal after the final sorting step were indeed JM109/pTX152, a sample of cells from the final round of FACS was plated on chloramphenicol plates and then replica plated on plates containing 100 μg/ml ampicillin. The pTX152 plasmid confers resistance only to chloramphenicol whereas the plasmid pTX101 which is present in the control cells also confers resistance to ampicillin. Over 95% of all the colonies examined were chloramphenicol resistant and ampicillin sensitive (cm⁺, amp⁻), consistent with the phenotype expected for JM101/pTX152 cells. As an additional test, plasmid DNA was isolated from eight cm⁺, amp-colonies and the presence of pTX152 was confirmed by restriction analysis.

The above results demonstrate that E. coli displaying a recombinant antibody on its surface can be recovered from at least a 10⁵-fold excess of control E. coli simply by incubation with a fluorescent hapten and fluorescent activated cell sorting. Only two rounds of sorting and regrowth of the sorted cell population are needed for enrichment from a large excess of background.

### Example 3

### Surface Expression of Anti-digoxin Antibody Incorporating Protease Cleavage Site

A construct similar to that used for surface expression of scFᵥ (digoxin) can be modified to incorporate a protease cleavage site. For example, the recognition sequence of enterokinase [(Asp)₄-Ile-Arg] can be introduced in the Lpp-OmpA(46-159)-scFᵥ between the OmpA(46-159) and the scFᵥ domains. The protease cleavage site at the N-temiinal of the scFᵥ antibody domain of the fusion protein is then used to release the scFᵥ antibody in soluble form following treatment of the cells with the appropriate proteolytic enzyme. Because the outer membrane of E. coli serves as a protective barrier to the action of externally added proteases, very few contaminating proteins will be present in the culture supernatant. A single colony expressing a desired single chain Fᵥ antibody can be grown in liquid media and harvested by centrifugation after overnight growth at 24° C. The cells are resuspended in buffer to maintain the pH approximately neutral. Protease added at appropriate concentrations to the fusion protein to be treated and incubated at least 4 hours at 4° C. will release the soluble single chain Fᵥ. Subsequently the cell suspension is centrifuged and the supernatant containing the solubilized single chain Fᵥ antibody is collected.

To separate the cleaved, soluble scFᵥ antibody from the protease, as well as any E. coli trace contaminants, a His₆ sequence may be introduced at the C-terminus of the scFᵥ using PCR amplification with the appropriate primers. Polyhistidine tails bind strongly to metals so that the fusion protein can be purified by immobilized metal-ion affinity chromatography (IMAC). The separation of the cleaved ScFᵥ antibody using is carried out as described by Georgiou et al. (1994).

Lpp-OmpA(46-159) fusions are typically expressed at a high level, typically around 5×10⁴ per cell. Thus the yield after protease treatment and IMAC is at least 2-3 mg of antibody per liter of shake flask culture.

### Example 4

### Solid Phase Immunoassays

This example illustrates a solid phase immunoassay using E. coli with anti-digoxin single chain Fᵥ displayed on the surface. This immunoassay is demonstrated to be a sensitive and quantitative technique. To facilitate the removal of the surface expressed antibody following the reaction with antigen, the cells may be attached to a solid support such as a membrane, dipstick or beads.

Digoxin-FITC (The Binding Site Inc. (San Diego, Calif.) was diluted to a concentration of 20 nM in PBS. Digoxin (Sigma, St. Louis, Mo.) was brought to a concentration of 1 μM in PBS. The pTX152/JM109 cells were grown in LB overnight at 37° C. then subcultured into fresh LB and grown overnight at room temperature. The cells were harvested and resuspended in PBS pH 7.4 at a concentration of 10¹⁰ cells/ml, based on the O.D.₆₀₀ to form a cell stock. Some cells were also resuspended in 15% glycerol/water and stored at 70° C. Frozen cells yielded the same results as freshly prepared cells.

The following reagents were transferred to a 1.5 ml microcentrifuge tube, 25 μl of 20 nM digoxin-FITC solution, 0.5-50 μl of 1 μM digoxin and PBS to a final volume of 950 μl. The mixture was vortexed briefly and pulsed in an Eppendorf raicrocentrifuge. A 25 μl or 100 μl aliquot of the cell stock (10¹⁰ cells/ml) was added to the mixture and allowed to incubate for 1 hour at room temperature. Following this incubation the cells were spun in a microcentrifuge for 5 minutes at 5,000 rpm and the supernatant collected. The fluorescence of the digoxin-FITC in the supernatant was measured using a fluorimeter.

This immunoassay was also performed with the cells attached to a solid support. The solid support was Fisher filter paper P5 was cut into strips approximately 0.5 cm×2 cm, and dampened in PBS by submerging one end of the filter paper and allowing it to move upward. The filter paper was allowed to dry slightly and 10 μl of a solution containing 6×10⁹ cells/ml was applied to the filter paper. The cells were fixed to the paper with a solution of 6-PLP, 8%PFA and NaIO₄. The 6-PLP solution is 10 mM 6-PLP, 200 mM MES, 700 mM NaCl, 50 mM KCl, 700 mM Lysine-HCl, 50 MM MgCl₂, and 70 nM EGTA followed by 0.01M MgCl₂ and 8% PFA. The 8% PFA was prepared by addition of 4 g of PFA into 50 ml of water, followed by heating the solution to 70° C. with constant stirring. Approximately 2 drops of a 1M solution of NaOH was added to the solution until it became clear. The solution was then filtered through Whatman filter paper #1, allowed to cool and stored at 4° C. until use. The final fixative solution was produced by mixing the components A, B and C to a final volume of 10 ml just prior to use, where A is 1 ml 6-PLP, 04 g sucrose and 3.03 ml water; B is 21.4 mg NaIO₄; and C is 4.62 ml 8% PFA. The fixative was applied dropwise to one end of the filter paper and allowed to soak upwards. The excess fixative was allowed to drain and the filter paper was washed twice with a solution of 100 mM NH₄Cl. The filter paper can then be stored in a solution of PBS until use.

The assay was performed by removing the filter paper with the cells attached and then measuring fluorescence of the solution.

Essentially quantitative assays for an antigen can be run using a mixture of a known amount of labeled antigen combined with an unknown amount of unlabeled antigen. In one exemplary study, pTX152/JM109 with antidigoxin antibody displayed on the surface were grown in LB overnight at 37° C., subcultured into fresh LB and grown overnight at room temperature. The cells were pelleted and resuspended in PBS to form a stock solution of cells at a concentration of 1×10¹⁰ cells/ml. A mixture of 25 μl of 20 nm digoxin-FITC and an amount of 1 μM free digoxin in the range of 0.5-50 μl, was prepared and brought to a final volume of 950 μl with PBS. A 25 μl or a 100 μl aliquot of the cell stock solution was added to the mixture to give a total of 0.25×10⁹ or 1×10⁹ cells. The mixture was then allowed to incubate at room temperature for 1 hour. The cells were pelleted and the fluorescence of the supernatant was measured for each sample.

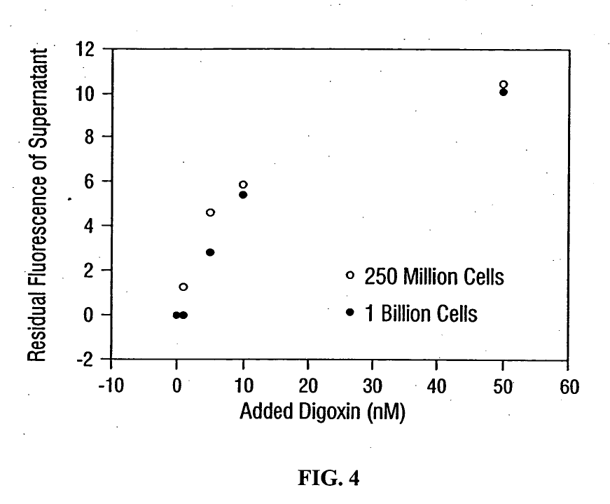

FIG. 4 shows a plot of the residual fluorescence observed with the indicated amount of free digoxin, 0.5 nM digoxin-FITC conjugate and either 250 million or 1 billion cells expressing the antidigoxin single chain antibody on their surface.

To facilitate the removal of the surface expressing antibody cells following the reaction, the cells may be attached to a solid support. In this example the solid support consisted of a membrane however many other supports would work as well such as dipsticks or beads.

Strips of filter paper were moistened with PBS and allowed to dry slightly. A 10 μl aliquot of a 6×10⁹ cells/ml of a cell suspension containing pTX152/JM109 cells displaying anti-digoxin antibodies on their surface was applied to the strips of pre-moistened filter paper. The cells were then fixed to the paper using a mixture of 6-PLP, PFA and NaIO₄, as described in the materials and methods, and washed twice with a solution of 100 mM NH₄Cl. Following the incubation no centrifugation is necessary the filter paper is simply removed from the solution and the residual fluorescence measured as described previously.

The assays described in this example use fluorescence as the indicator of binding; however, other indicator reactions may be used such as radioactivity, enzyme conjugates and when the surface expressed antibody is catalytic, an assay for catalytic activity may be used.

### Example 5

### Discrimination of Surface Displayed Antibody Affinity by Flow Cytometry

This example demonstrates that the display of antibodies on the surface of microorganisms constitutes the basis for discriminating between antibodies of different affinities. By adjusting the fluorescent antigen concentration, it is possible to discriminate binders of high affinity from those with moderate affinity.

The heavy-chain residue Y33 of the scFv(digoxin) antibody is known to be critical for antigen binding (Short et al., 1995). The following mutants of the scFv (digoxin) antibody: Y33N, Y33C, Y33S, Y33G, Y33STOP were constructed by overlap extension PCR™ (Ho, 1989). The large fragment from EcoR I digested pSD192 was purified and religated. to yield pSD195 (AmpR). Primers Y33C.s, Y33N.s, Y33S.s Y33G.s, and Y33Stop.s along with 3′ primer CMI.5 were used to amplify 3′ fragments for mutant constructions. The 5′ overlap fragment was produced with primers #4 and #3 and overlapped with the mutant fragments using primers #4 and CMI.5. The resulting products were digested with EcoRI and ligated into pSD195, and electroplated into JM109 to yield Cm resistant mutants pY33N, pY33C, pY33S, pY33G, and pY33STOP. Overnight cultures were grown at 37° C. and subcultured 1:100 at 25° C. for 20 hrs. cells (200 ml) were harvested into 1 ml PBS pH 7.1-7.4, pelleted by centrifugation at 3000× g for 4 min, and resuspended in 1.5 ml PBS. Cells were aliquoted into a clean eppendorf tube and fluorescein-conjugated digoxin (The Binding Site, Eugene, Oreg.) was added to 1×10⁻⁷ M. Cells were incubated at room temperature (22-24° C.) for 1 hr with gentle shaking, pelleted and resuspended in 1 ml and analyzed by flow cytometry. At least 10,000 events were acquired on a Becton Dickinson (San Jose, Calif.) FACSort. Parameters were set in LOG mode as follows: FSC threshold 80, FSC preamp E01. FL1 800, SSC 400. Debris and other particulate material were excluded by defining an appropriate FSC-SSC around the cell population.

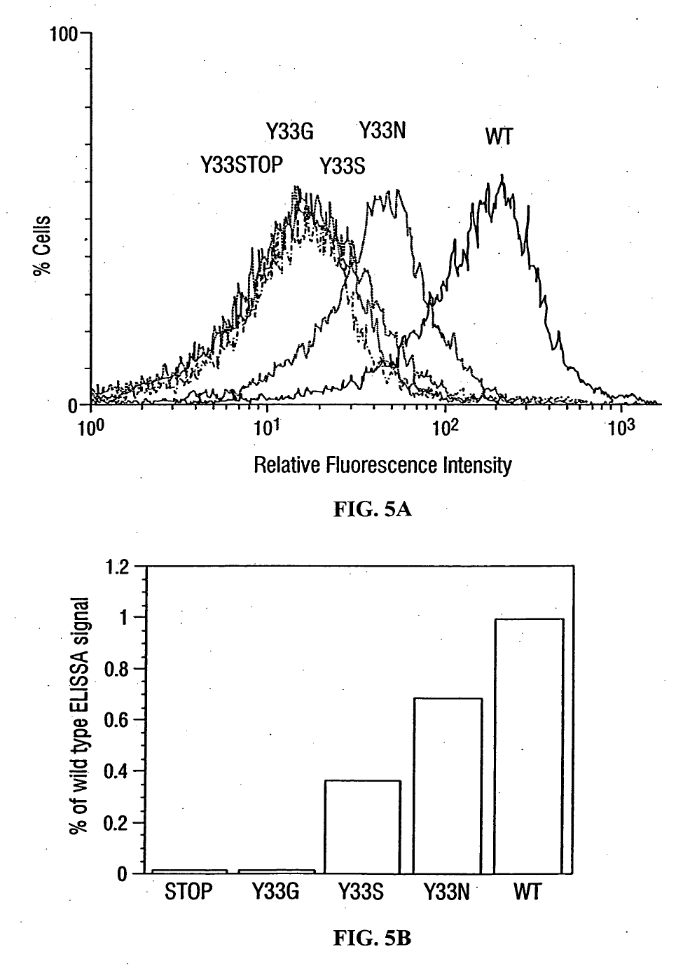

The relative affinity of the mutants constructed were, as determined by in vitro ELISA data, Y33N (moderate), Y33S (low), Y33G (background), and Y33Stop (background). The flow cytometry data and previously obtained ELISA results (Burks et al., 1997), are shown in FIG. 5A and FIG. 5B, respectively. Importantly, the flow cytometry data for individual Y33 mutants correlate with ELISA data. In particular, the mean fluorescence signals vary in the manner WT>Y33N>Y33S>Y33G_Y33Stop. The results further demonstrate that the high affinity wild-type scFv may be discriminated from the low affinity Y33S, and to a lesser extent, from the moderate affinity Y33N at concentrations of fluorescent-digoxin approximately 20-fold greater than the dissociation constant.

### Example 6

### Flow Cytometric Assay for the Determination of Antigen Binding Affinities of Antibodies Displayed on Cell Surfaces

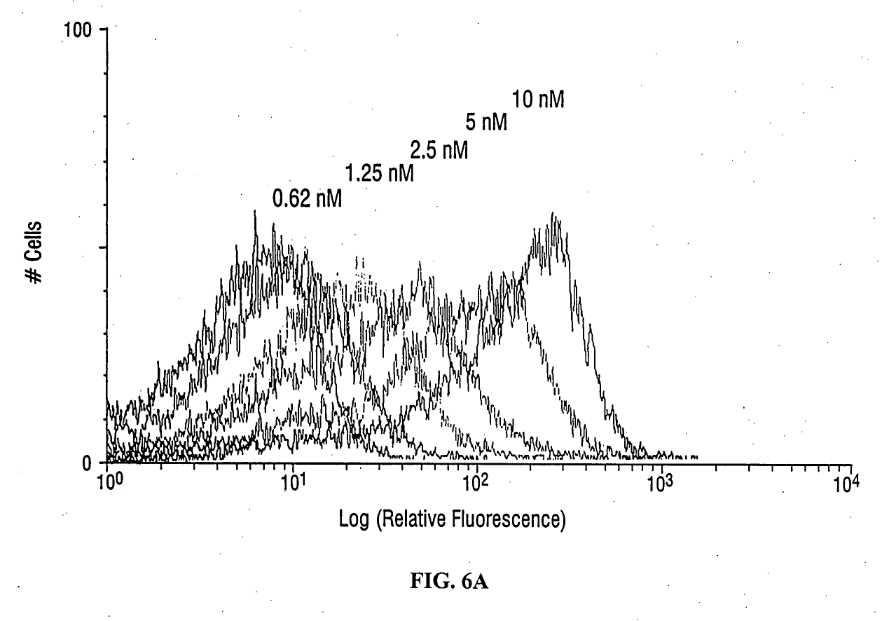

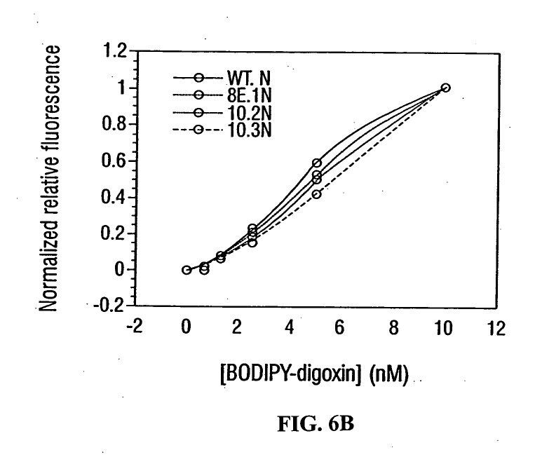

The availability of a fluorescent-antigen conjugate affords a simple and rapid method for flow cytometric affinity estimation of antibodies displayed on the cell surface. In this example cell aliquots were incubated with various concentrations of BODIPY-digoxin, diluted in PBS, and the mean total fluorescence was determined by flow cytometry. For detailed flow cytometric analysis, 20 ml were added directly from a 24 hr, 25° C. culture to 180 ml BODIPY-digoxin at 10, 5, 2.5, 1.25, 0.62 and 0 nM in 96-well-plate, and allowed to incubate 1 hr with gentle shaking. Cell samples were diluted 1:5 in PBS for flow cytometric analysis. Mean fluorescence was recorded for each sample, and the resulting data were used to plot a saturation curve and calculate the relative dissociation constant. Fluorescence histograms for the wild-type antibody expressing cells over a range of concentrations is shown in FIG. 6A. Normalized saturation curves for three mutants with apparent affinities within 1.5 fold of the wild type are shown in FIG. 6B.

### Example 7

### Screening Antibody Libraries by Cell Surface Display and Fluorescence-Activated Cell Sorting

Vector Construction for Surface Expression of scFv Libraries

The successful construction and screening of polypeptide libraries displayed on the cell surface it was found to be important to construct novel vectors that eliminate the probability of contamination of the library with wild-type plasmid molecules.

Thus, pSD195 containing only the N-terminal portion of the cat gene immediately downstream of the scFv to be expressed at the cell surface, was constructed. Two Pst I restriction sites were introduced, by silent mutagenesis, into the vector to simplify library construction. The first site was created just upstream of the scFv LCDR3 to be randomized and the second was introduced within the chloramphenicol resistance gene, (CmR). Removal of the Pst I insert followed by religation generated pSD195, which does not allow host growth in the presence of chloramphenicol (Cm), since cat is functionally inactivated. Thus, after library ligation and transformation, only clones that contain the correct PCR™ generated insert, in the correct orientation, will restore Cm resistance, and consequently, growth in the presence of Cm.

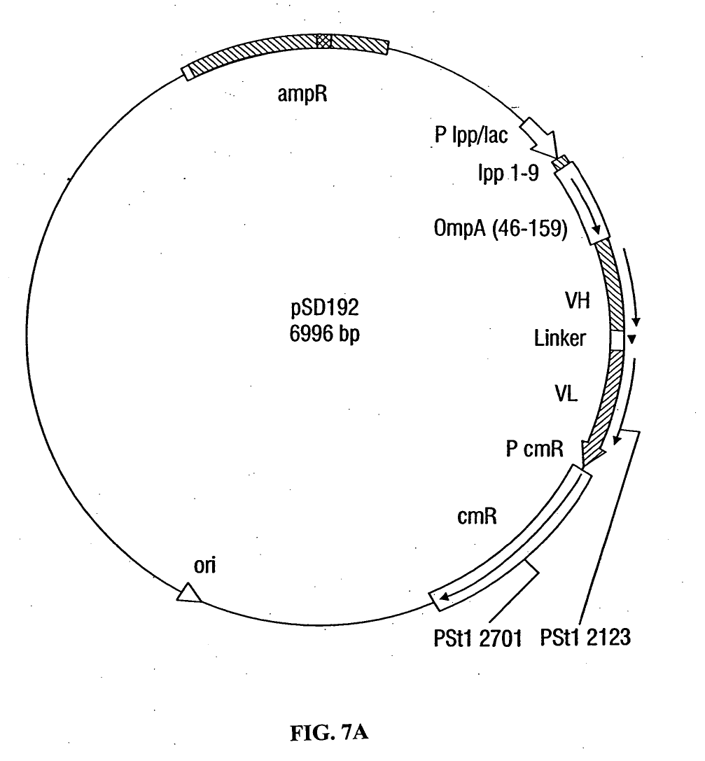

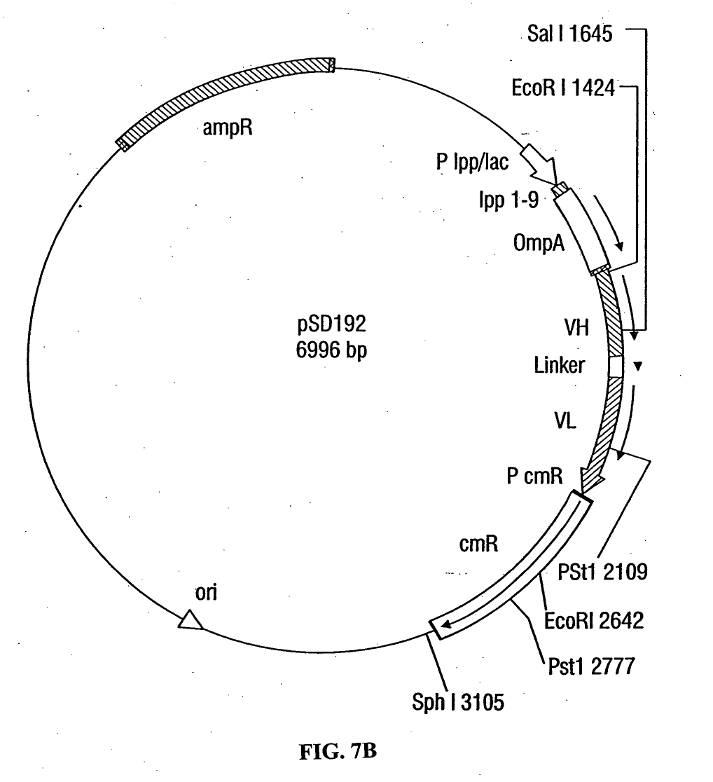

All DNA manipulations were as described elsewhere (Maniatis, 1996). Restriction enzymes were from Promega (Madison, Wis.), and Pfu polymerase from Stratagene (La Jolla, Calif.). Escherichia coli strain JM109 was used for all cloning steps and library experiments. Oligonucleotides for PCR™ reaction are shown in Table 4. Overnight cultures were grown at 37° C. in LB media supplemented with 0.2% glucose, ampicillin (100 μg/ml) and chloramphenicol (30 μg/ml), and subcultured 1:100 at 25° C. for a specified duration. pTX152 was digested with Pvu I and BamH I, and the small fragment from pTX152 was ligated into similarly digested pET-22b to yield pGC183. The chloramphenicol acetyl transferase gene (cat) was amplified from pBR325 with primers CM.1.s and CM.2.as, digested with EcoR I and Sph I and ligated into similarly digested pGC183 to yield pGC185. Finally, the digoxin scFv was reintroduced into pGC185 by ligating the EcoR I-BamH I fragment from pTX152 with similarly digested pGC185 to yield the pGC182 (ampR, cmR). Subsequently, the Pst I site was removed from pGC182 by replacing the AlwN I/Pvu I fragment with that from pUC18 giving rise to pSD182. Pst I sites were introduced upstream of the light-chain CDR3 and within the cat gene by silent mutagenesis. Primers #4 and PIA.1as (Rxn1), PIA.2s and PIB.1as (Rxn2), and PIB.2s and SphI.2.as (Rxn3) were used to amplify the corresponding fragments from pTX152, pSD182, and pBR322 respectively. Products from Rxn2 and Rxn3 were amplified with PIA.2 and Sph1.2as and the resulting product was overlapped with the Rxn 1 product using primers #4 and SphI.2as, digested with EcoR I and Sph I and ligated into similarly digested pSD182 to yield pSD192 (FIG. 7).

TABLE 4
OLIGONUCLEOTIDE PRIMERS FOR LIBRARY CONSTRUCTION

| | | |
|---|---|---|
| #4 | TGGACCAACAACATCGGT | SEQ ID NO:3 |
| CMI5 | CCCATATCACCAGCTCACCGTCTTTC | SEQ ID NO:4 |
| CMI4 | GACCCCGAGGACTAACGTCTTCGAATAAATAC | SEQ ID NO:5 |
| CM.1 | CCGAATTCGTTTGAACATGCCTAAC | SEQ ID NO:6 |
| CM.2 | CGGAATTCGTGCGCAACACGATGAAGCTC | SEQ ID NO:7 |
| SPH1.2.AS | AGGGCATGCAAGGGCACCAATAACTGCCTTA | SEQ ID NO:8 |
| P1A.1.AS | TTGGCTGCAGTAATATATTGCAGCAT | SEQ ID NO:9 |
| P1A.2.S | TGCAATATATTACTGCAGCCAAACTACGCAT | SEQ ID NO:10 |
| P1B.2.S | CGGCAGTTTCTGCAGATATATTCGCAAGAT | SEQ ID NO:11 |
| P1B.1.AS | CTTGCGAATATATCTGCAGAAACTGCCGGAA | SEQ ID NO:12 |
| P1B.AS | ACGCCACATCTTGCGAATATATCTGCAGAAACTGCCGGAA | SEQ ID NO:13 |
| #3 | CAGGGTACATTTTCACCG | SEQ ID NO:14 |
| LCDR3.S | AACTGCAGCCAANNBACGCATNNBCCANNBACGTTCGGCTCGGGGA | SEQ ID NO:15 |
| HCDR3.1.S | GTATACTATTGCGCCGGCTCCTCTGGTAACNNSNNSNNSNNSGATTATTGGGGTCATGGTGCT | SEQ ID NO:16 |
| H2.AS | GTTACCAGAGGAGCCGGCGCAATAGTATAC | SEQ ID NO:17 |
| Y33N.S | TACATTTTCACCGACTTCAATATGAATTGGGTTCGC | SEQ ID NO:18 |
| Y33C.S | TACATTTTCACCGACTTCTGCATGAATTGGGTTCGC | SEQ ID NO:19 |
| Y33S.S | TACATTTTCACCGACTTCTCTATGAATTGGGTTCGC | SEQ ID NO:20 |
| Y33G.S | TACATTTTCACCGACTTCGGGATGAATTGGGTTCGC | SEQ ID NO:21 |
| Y33STOP.S | TACATTTTCACCGACTTCTAAATGAATTGGGTTCGC | SEQ ID NO:22 |

Construction of Cell Surface Displayed scFv Libraries

The digoxin scFv light-chain codons for T91, V94, and P96 were chosen for randomization. Both T91 and P96 form important contacts with digoxin in the Fab crystal structure (Jeffrey et al., 1993), suggesting that they play an important role in determining the antibody affinity.

Light-chain residues T9 1, V94 and P96 were randomized by PCR™ with Pfu polymerase (Stratagene, La Jolla, Calif.). Primers (Genosys, The Woodlands, Tex.) LCDR.3.s and CMI.5 were used to amplify the LCDR3-cmR 700 bp fragment encoding the antibody LCDR3 and cat gene fragment with non-Pst I containing pSD182 as a template to prevent wild-type contamination. The resulting PCR™ product was digested with Pst I, purified by gel electrophoresis and electroelution and ligated into Pst I digested, phosphatase treated pSD195. The resulting plasmid library was electroporated into JM109 to yield 2×10⁵ transformants. After an 8 hr growth in liquid media, light-chain library plasmid DNA was prepared to provide a stock for subsequent library screening experiments. The probability that each amino acid sequence is represented in the library pool may be calculated, using a Poisson distribution (Lowman et al., 1991), to be greater than 85%. Flow cytometric analysis of ten randomly picked clones showed 1/10 to weakly bind fluorescein-digoxin and 9/10 clones exhibited no binding.

A second library was created by randomizing heavychain residues H99.K, H100.W, H100a.A, and H100b.M) using the NNS (S=G or C) randomization scheme by overlap extension PCR (Ho, 1989). pSD182 was digested with Xba I and the large fragment was purified by gel electrophoresis, and religated to yield pSD181. pSD181 was digested with Xba I and Apa I, and the small fragment was gel purified and used as a template to produce the 3′ mutagenic antibody-cat fragment. The 5′ fragment was amplified from pTX152 with primers EcoRI.s and H3.as. After gel purification, 5′ and 3′ fragments were subjected to overlap extension PCR with primers #4 and PIB.as. PCR master mix (150 ml 10× Pfu buffer, 200 μM dNTPs, 8 ng/uL each primer, 1 ng/uL 5′ and 3′ template fragments and Pfu (0.03 u/mL and water to 1.5 ml) was transferred in 100 ml aliquots into thin-wall eppendorf tubes and amplified as follows: 1 cycle 3 min (94° C.), 3.5 min (49° C.). 3 min (76° C.), 30 cycles 2 min, 3.5 min, 3.5 min, 1 cycle 7 min (76° C.). Products were combined, precipitated with ethanol, resuspended in 500 uL TrisHCl (10 mM) pH=8.5, and purified by gel electrophoresis and electroelution. HC PCR DNA was digested with Pst I, precipitated, digested with Sal I, gel purified and electroeluted. pSD195 was digested with Sal I and Pst I Gel purified and electroeluted. Ligations (12 ug pSD195 Sal I/Pst I, 3.6 ug HC PCR Sal I/Pst I, 40 μl ligase, 80 μl 10×, water to 800 μl) were incubated 20 hr at 16° C., heat inactivated 10 min at 70° C., ethanol precipitated, and resuspended in 60 ul Tris-HCl. Four aliquots of 15 ul were individually electroporated into 300 ul electrocompetant JM109 cells. Electroporation cuvettes were washed with 3 ml of SOC media, and incubated for 1 hr at 37° C. in a total volume of 60 ml. Prewarmed LB media (500 ml) supplemented with 0.4% glucose, and 75 mg/ml ampicillin was added and 1:10 and 1:100 dilutions were plated on LB plates supplemented with Cm (30 μg/ml) and Amp (100 μg/ml). Plating of dilutions revealed the library to contain 4×10⁶ individual transformants. After 7 hrs of growth the culture was subcultured 1:50 into 500 ml LB, Amp (100), Cm (30), 0.2% glucose, and grown for 8 hr. Plasmid DNA was isolated to provide as a stock for HC library experiments. Flow cytometric analysis of the same twenty clones showed weak binding for three clones at 100 nM fluorescein-digoxin, and 17/20 clones displayed levels of fluorescence consistent with background or autofluorescence.

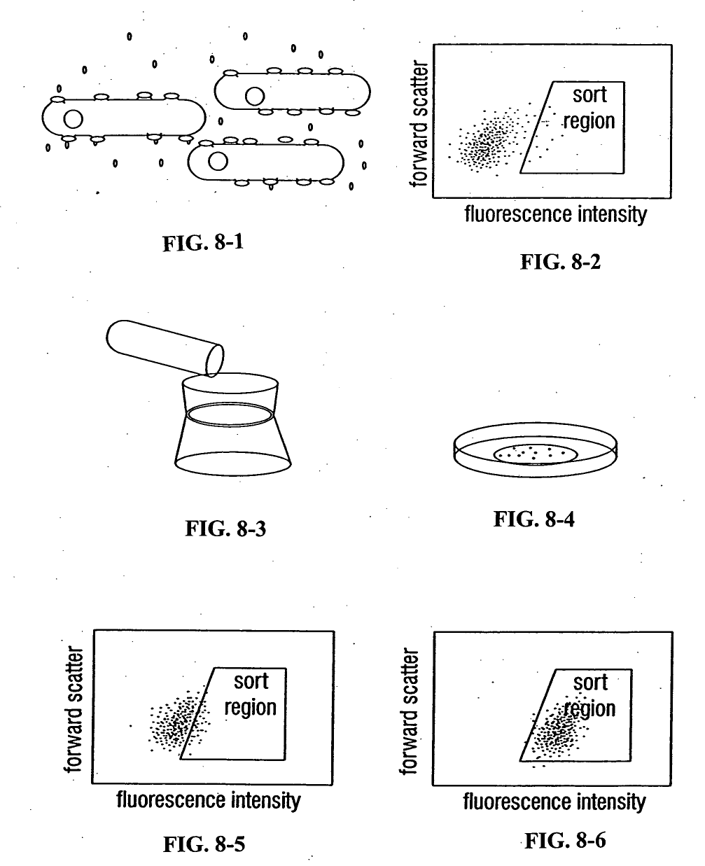

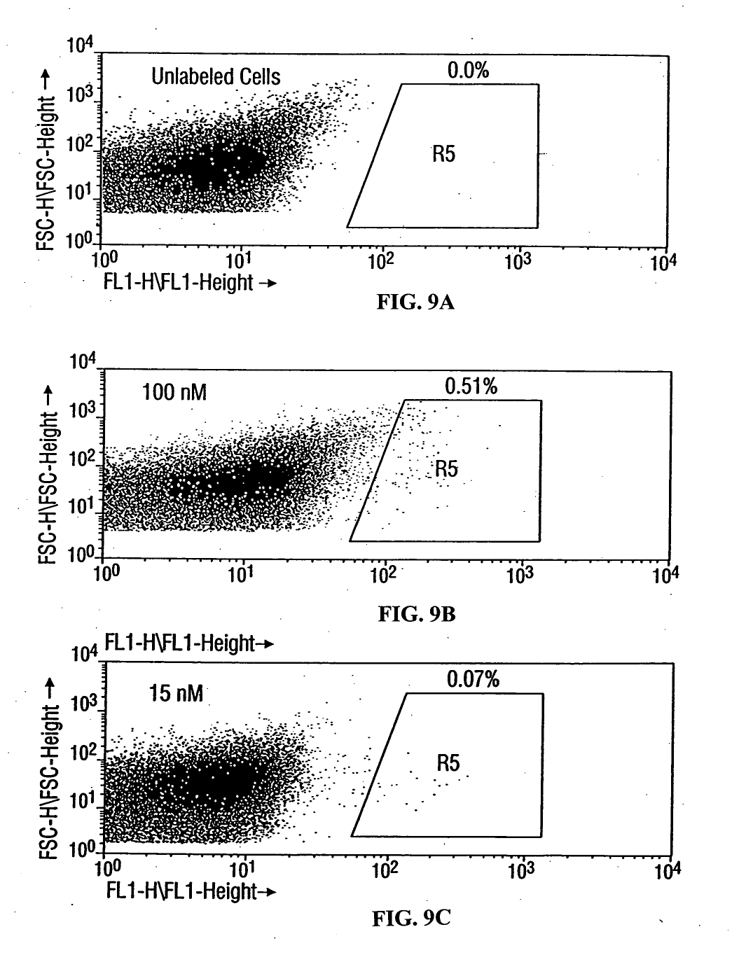

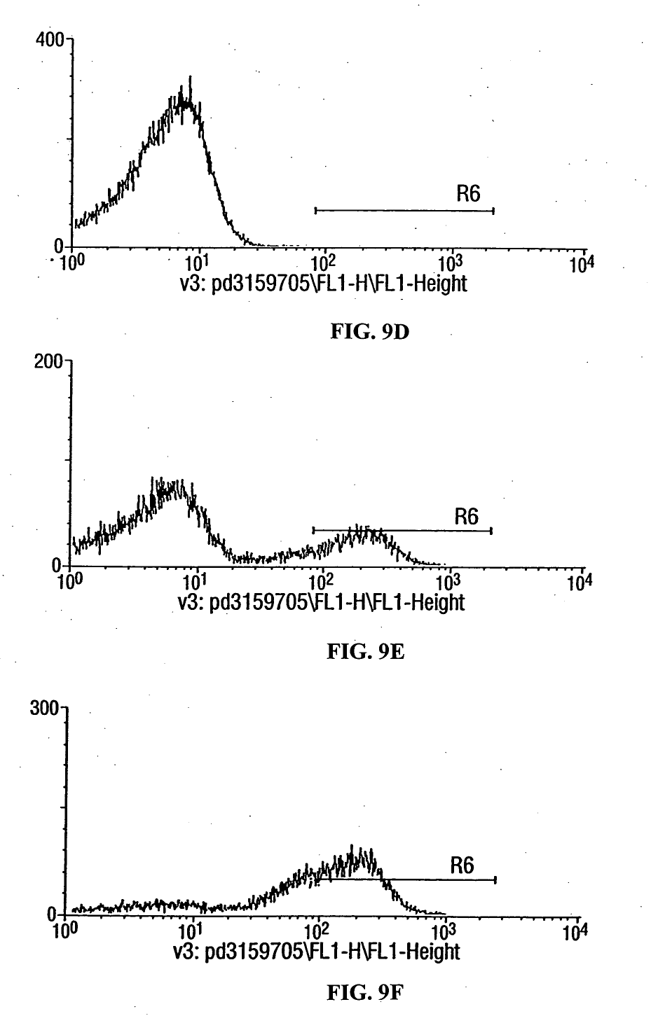

Clones that bind to the hapten digoxin were isolated from the library in a single step. This procedure is outlined in FIG. 8. Following incubation of a library aliquot with fluorescein-digoxin, cells displaying a total fluorescence greater than threshold value, which the user may gradually decrease to reduce selection stringency, were sorted and recovered by vacuum filtration onto a 0.2 μm membrane (Millipore, Bedford, Mass.). The membrane was transferred to an agar plate containing the appropriate antibiotics and incubated overnight at 37° C. Individual colonies were then grown in liquid media and assayed for high-affinity antigen binding by flow cytometry. Light-chain library DNA (3 μL) was transformed into JM109 by electroporation. Following a 12 hr subculture at 25° C., 200 ul of the library culture was diluted into 1 ml of sterile PBS, pelleted in an microcentrifuge at 3000 g for 5 min, and resuspended in 1.5 ml PBS. Library aliquots were incubated in the dark, with shaking, at 25° C. with fluorescein-digoxin tracer (10⁻⁷, 10⁻⁸, 10⁻⁹ M) for 1 hr, pelleted and resuspended in PBS at approximately 10⁸ cells/ml for flow cytometric analysis or sorting. 20,000 events were acquired for each concentration (FIG. 9A-FIG. 9F). Highly fluorescent cells were asceptically sorted in exclusion mode and filtered onto a 0.2 μm cellulose acetate membrane (Millipore, Bedford, Mass.). The filter was transferred to an agar plate and incubated overnight at 37° C. Individual colonies were picked and grown at 37° C. to saturation, subcultured at 25° C. and grown 18 hr post selection analysis. Cells were labeled as described above with fluorescent digoxin tracer (1×10⁻⁷ M) and analyzed by flow cytometry. The amino acid sequences of clones displaying high fluorescence were determined by DNA sequencing and the affinity was characterized by flow cytometry. The combined results of two library sorting experiments are summarized in Table 5.

TABLE 5
ScFv (dig) VARIANTS ISOLATED FROM A
LIGHT-CHAIN LIBRARY BY FACS

| | | | Relative |
|---|---|---|---|
| Clone | LCDR3 | | Fluorescence |
| WT | SQTTHVPPT | SEQ ID NO:23 | +++ |
| LC1-1 | SQATHMPGT | SEQ ID NO:24 | +++ |
| LC1-2 | SQTTHFPVT | SEQ ID NO:25 | +++ |
| LC1-3 | SQATHYPTT | SEQ ID NO:26 | +++ |
| LC2-3 | SQCTHWPVT | SEQ ID NO:27 | +++ |
| LC2-4 | SQTTHVPPT | SEQ ID NO:28 | ++ |
| LC2-2 | SQATHYPST | SEQ ID NO:29 | +++ |
| LC2-6 | SQATHSPST | SEQ ID NO:30 | +++ |
| PRESORT | | | |
| LC-1 | SQVTHGPRT | SEQ ID NO:31 | + |
| LC-2 | SQGTHRPYT | SEQ ID NO:32 | + |
| LC-3 | SQITHVPKT | SEQ ID NO:33 | + |
| LC-4 | SQLTHLPRT | SEQ ID NO:34 | |
| LC-5 | SQPTHVPPT | SEQ ID NO:35 | |
| LC-6 | SQVTHKPGT | SEQ ID NO:36 | − |
| LC-7 | SQLTHWPST | SEQ ID NO:37 | − |
| LC-8* | SQLTHGPRT | SEQ ID NO:38 | − |
| LC-9* | SQLTHGPRT | SEQ ID NO:39 | + |
| LC-10 | SQZTHGPFT | SEQ ID NO:40 | − |

*LC-8 and LC-9 are unique at the DNA level
+++ = 100 < mean fluorescence (10⁻⁷ M)
++ = 60 < mean fluorescence <100
+ = 30 < mean fluorescence <60
− = < mean fluorescence <30

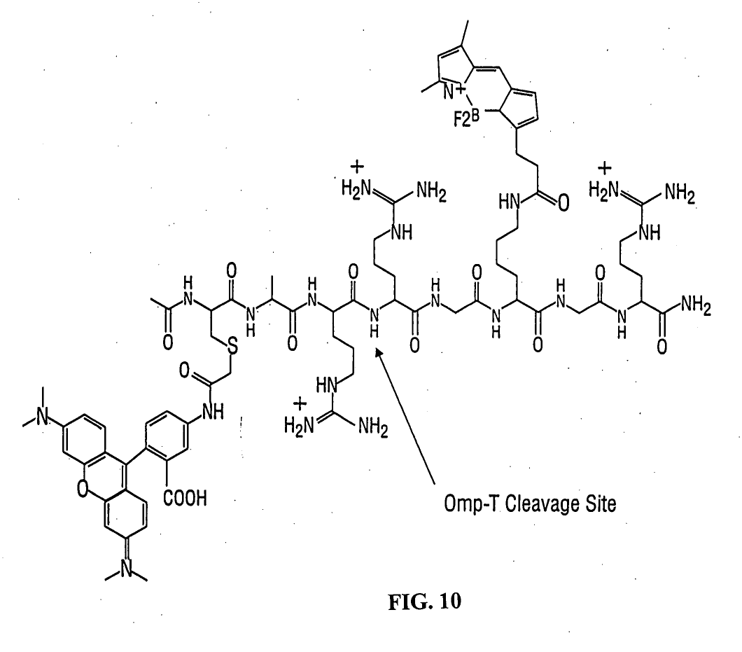

The heavy-chain library was first screened in a single pass essentially as described for the light-chain library, to demonstrate that high affinity scFv(dig) variants could be selected from large libraries using only a single FACS step. HCDR3 library DNA (3 ml) was electroporated into JM109 and grown 8 hr to saturation at 37° C. After a 14 hr subculture at 25° C., 160 μl cells were harvested into 640 μl of 100, 15, and 0 nM BODIPY-digoxin in PBS. Cells were incubated with gentle shaking for 45 min. Propidium iodide was added to a final concentration of 5 μg/ml and cell were incubated 15 min. Cells were washed twice with PBS and resuspended to give a FACSort event rate of 1000 s⁻¹. Data from 50,000 events was acquired for each concentration (FIG. 10A). After sterilization of the FACSort with 70% ethanol, a total of 10⁷ cells were sorted in recovery mode and 405 events were collected. Sorted cells were filtered onto a 0.7 μm membrane (Millipore, Bedford, Mass.) and transferred to a petri dish containing a sterile presoaked petri pad (Millipore, Bedford, Mass.) and allowed to incubate overnight at 37° C. 192 colonies were picked with a pipette tip and grown in 200 μl in 96-well plates overnight. Cells were subcultured 1:100 and grown at 22-24° C. for 20 hr. In a fresh 96-well plates 10 μl cells was added to 90 μl of 18 nM BODIPY-digoxin. Cells were incubated 1 hr and diluted into 500 ml PBS total for flow cytometric analysis. A total of 400 selected clones were picked and grown in 96-well plates for a preliminary flow cytometric analysis. 95/190 exhibited mean fluorescence greater than 100. Six clones did not grow. Plasmid DNA was purified from 40 clones exhibiting the highest levels of fluorescence (mean fluorescence intensity >150). Plasmid DNA was digested with Nco I to prevent unnecessary analysis of clones possessing the wild-type restriction site CGGATC. DNA sequencing results are shown in Table 6.

TABLE 6
HEAVY-CHAIN LIBRARY SCREENING RESULTS

| Clone | HCDR3 | | K_{d}/K_{Dwt} |
|---|---|---|---|
| WT | SSGNYRALDY | SEQ ID NO:41 | 1 |
| HC1.4 | SSGNYRALDY | SEQ ID NO:42 | 2. |
| HC10.3 | SSGNRRAWDY | SEQ ID NO:43 | 1. |
| HC10.2 | SSGNRRALDY | SEQ ID NO:44 | 1. |
| HC8.1 | SSGNGRAWDY | SEQ ID NO:45 | 1. |
| HC1.3 | SSGNISALDY | SEQ ID NO:46 | >10 |
| HC2.1 | SSGNQRKMDY | SEQ ID NO:47 | >5 |

TABLE 7
LCDR3 LIBRARY SPECIFICITY SORT SEQUENCES

| Clone | LCDR3 | | Relative FL1 |
|---|---|---|---|
| WT | SQTTHVPPT | SEQ ID NO:48 | +++ |
| LG2-5 | SQVTHRPLT | SEQ ID NO:49 | + |
| LC2-7 | SQVTHDPGT | SEQ ID NO:50 | + |
| LC3-1 | SQVTHCPST | SEQ ID NO:51 | ++ |
| LC3-7 | SQVTHWPPT | SEQ ID NO:52 | +++ |
| LC3-10 | SQVTHYPVT | SEQ ID NO:53 | + |

+++ = 100 < mean fluorescence (10⁻⁷ M)
++ = 60 < mean fluorescence < 100
+ = 30 < mean fluorescence < 60
− = < mean fluorescence < 30

The heavy-chain library was also screened using a multiple-step FACS process to enrich high-affinity antibody expressing cells. A library aliquot was labeled with BODIPY-digoxin at 70 nM, and sorted at 1200 s⁻¹. Cells were sorted directly into supplemented LB media and grown with shaking at 37° C. for 15 hrs. Cells were subcultured for 14 hrs and relabeled with 5 nM BODIPY-digoxin. Cells were sorted a second time in exclusion mode and again amplified by growth. The final population was relabled with 5 nM BODIPY-digoxin and analyzed by flow cytometry. Fluorescence histograms for pre-enriched, as well as single and double-enriched populations are shown in FIG. 10B.

Selection for Altered Specificity

Selection based upon specificity was accomplished by preincubating the light chain library with the digoxin analog, digitoxin, and then subsequently labeling the preincubated cells with fluorescein-digoxin. An aliquot of the LCDR3 library was incubated with digitoxin (5×10⁻⁶ M) for 30 min at 25° C. with steady gentle inversion. Fluorescein-digoxin was added to 10⁻⁷ M and cells were incubated an additional hour. Cells were pelleted by centrifugation and resuspended to give a FACS event rate of 1000 s⁻¹. A total of 10⁶ cells were screened and cells displaying high fluorescence were collected by FACS. Colonies were picked grown for FACS analysis. Plasmid DNA was prepared from clones displaying a mean fluorescence greater than 50 for DNA sequencing analysis. Sequencing analysis of five clones displaying high fluorescence showed a total consensus for the amino acid valine at wild-type position L91 (Table 5). Ten of ten clones selected in the absence of digitoxin preincubation did not have a valine residue at that position.

### Example 8

### Isolation of Cells Displaying Enzymes From a Vast Excess of Cells That do not Express an Active Enzyme

This example demonstrates that the enzymatic activity, rather than a ligand binding activity, of a surface displayed polypeptide can be used to isolate cells producing such a surface enzymatic activity from a vast excess of cells that do not express enzymatically active polypeptides. The example specifically demonstrates how E. coli cells that express the protease OmpT on their surface can be distinguished from cells that do not produce OmpT or produce inactive OmpT.

A substrate that becomes fluorescent upon cleavage by OmpT was designed by conjugating a fluorescent dye (BODIPY) and a quenching group (trimethylrhodamine) at the opposite ends of the secile bond. Enzymatic cleavage releases the quenching group into the medium resulting in the production of a fluorescent product. The fluorescent product was designed to have several positive charges to allow its binding to the surface of the cells via electrostatic interactions with the negatively charged lipopolysaccharide molecules that comprises the outer layer of the E. coli surface. The chemical structure of the substrate is shown in FIG. 10.

The peptide moiety was synthesized at the University of Texas, Austin peptide facility using standard FMOC coupling conditions. The thiol of the cysteine was alkylated with trimethylrhodamine iodoacetamide and this product was acylated with BODIPY-FL succinamidyl ester (Molecular Probes). The crude product was purified by preparative HPLC.

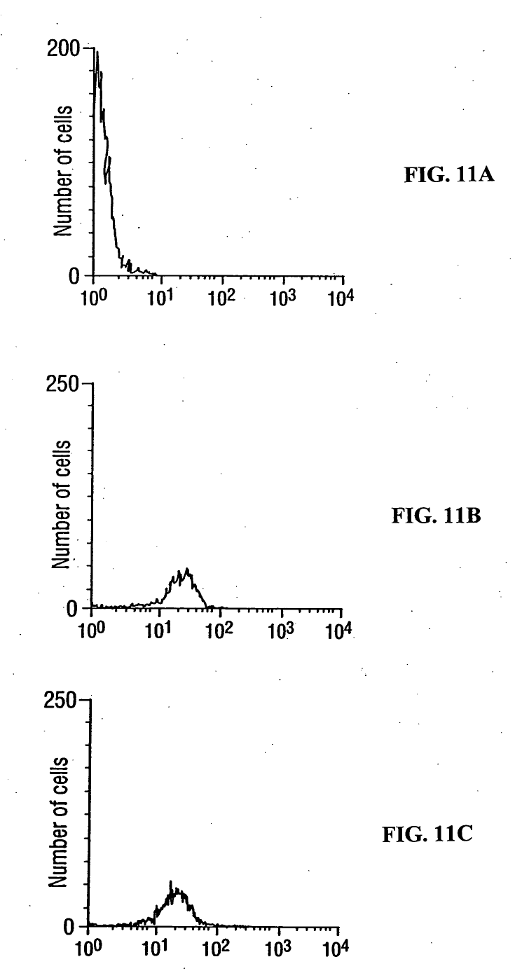

Different strains of bacteria were exposed to the substrate for 10 min. and examined by FACS. The OmpT negative E. coli mutant UT5600, shows no fluorescence (FIG. 11A). However, UT5600 cells expressing OmpT from a multicopy plasmid (pML19), showed a much larger increase in fluorescence, which continued to increase for over 20 minutes. The mean fluorescence intensity of the OmpT⁺ cells was over 30 times higher than that of the cells without the plasmid (i.e., OmpT cells). Such a difference in OmpT fluorescence is more than sufficient to allow the sorting of cells expressing active enzyme from cells that do not express OmpT (FIG. 11).

It was demonstrated that inactive OmpT mutants exhibit increased fluorescence upon incubation with the substrate. For this purpose, an OmpT mutant was produced in which the conserved His212 residue (which is thought to be part of the catalytic triad) was converted to Ala by site-directed mutagenesis (Maniatis et al. 1989). The His212->Ala mutant was confirmed to have no OmpT activity. UT5600 cells expressing the His212->Ala produce the same amount of OmpT as cells transformed with a plasmid encoding the wild type enzyme. When UT5600 cells expressing the inactive OmpT mutant were incubated with the substrate and examined by FACS they exhibited a background fluorescence that could be clearly distinguished from that of OmpT positive cells.

In other studies it was demonstrated that OmpT⁺ cells can be readily isolated from a population containing a huge excess of OmpT⁻ cells. Specifically, OmpT⁺ cells were mixed with OmpT⁻ cells at a 5,000-fold excess. The cell mixture was incubated with the substrate, passed through the fluorescence activated cell sorter and cells exhibiting a high fluorescence intensity were isolated. Nine out of nine sorted clones that were isolated produce OmpT. These studies showed that the OmpT⁺ cells can be readily isolated by FACS from a huge excess of background cells solely on the basis of the enzymatic activity of the OmpT protease (FIG. 12).

### Example 9

### Isolation of Clones Expressing Desired Enzymes From Combinatorial Polypeptide Libraries Displayed on the Cell Surface

This example illustrates how libraries of polypeptides displayed on the cell surface can be screened to isolate clones that produce polypeptides having a desired enzymatic activity. Specifically this example teaches the screening of libraries of OmpT mutant polypeptides to isolate novel enzymes that can hydrolyze peptide sequences not recognized by the wild-type OmpT enzyme.

The ompT gene will first be subjected to random mutagenesis using established techniques such as error-prone PCR, chemical mutagenesis or mutator strains. For error-prone PCR or chemical mutagenesis the ompT gene is first excised from the high copy plasmid pML19 by digestion with appropriate restriction endonucleases and mutagenized according to standard procedures known to those skilled in the art (Maniatis et al. 1989, Innis et al. 1990). Following mutagenesis, the ompT DNA will be ligated back to restriction-enzyme digested pML19 and the ligation mixture will be electroporated into E. coli UT5600. Transformants will be grown in LB broth containing ampicillin (100 μg/ml) and glucose at 0.2% w/v at 37° C. Cultures will be grown to saturation to ensure maximal expression of OmpT. Subsequently, the cells will be harvested by centrifugation, washed with PBS and resuspended in 1 mM Tris buffer, pH 7.0, in the presence of a substrate having a structure similar to the one shown in FIG. 10 except that the Arg-Arg dipetide that is recognized by the wild type OmpT is substituted with other dipeptide sequences, for example Arg-His, Arg-Ala, His-His, etc. Cells expressing OmpT capable of hydrolyzing the substrate allow the release of the trimethylrhodamine quencher into the solution while the N-terminal cleavage product containing the BODIPY fluorophore is electrostatically retained by the cells. As a result, cells displaying mutant OmpT proteins capable of hydrolyzing the substrate will become fluorescent and can thus be isolated by fluorescent activated cell sorting (FACS). For the isolation of enzymatically active cells by FACS, a gate is set such that only cells exhibiting high fluorescence are sorted in the positive window. Cells will be sorted at an event rate of at least 1000 s⁻¹. A total of 10⁶ cells will be screened and cells displaying high fluorescence will be collected by FACS. Isolated colonies will be then screened for product hydrolysis by FACS analysis. Finally, the DNA sequence of the mutant ompT genes encoding enzymes with altered substrate specificity will be determined by DNA sequencing.

All of the composition and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

J. References

The following references, to the extent that they provide exemplary procedural or other details supplementary to those set forth herein, are specifically incorporated herein by reference.

Agterberg, et al., “Outer-membrane PhoE protein of Escherichia coli K-12 as an exposure vector: possibilities and limitations”, Gene 88:37-45, 1990

Aiyar & Leis, “Modification of the Megaprimer Method of PCR Mutagenesis: Improved Amplification of the Final Product,” BioTechniques, 14:366-369, 1993

Antibodies: A Laboratory Manual, Cold Spring Harbor Laboratory, 1988

Baneyx and Georgiou, J. Bacteriol., 172:491-494, 1990.

Barbas et al., “Assembly of combinatorial antibody libraries on phage surfaces: The gene III site,” Proc. Natl. Acad. Sci. USA, 88:7978-7982, 1991

Barbas et al., “Semisynthetic combinatorial antibody libraries: A chemical solution to the diversity problem,” Proc. Natl. Acad. Sci. USA, 89:4457-4461, 1992

Better et al., “Escherichia coli Secretion of an Active Chimeric Antibody Fragment,” Science, 240:1041-1043, 1988

Blackburn et al., “Electrochemiluminescence Detection for Development of Immunoassays and DNA Probe Assays for Clinical Diagnostics,” Clinical Chemistry, 37(9): 1534-1539, 1991

Boder, E. T. and Wittrup, K. D., “Surface display of a functional single chain Fv antibody in Saccharomyces cerevisiae” Submitted 1996

Brennan, Chemical and Eng. News, 74:31033, 1996

Brown, S., “Engineered iron oxide-adhesion mutants of the Escherichia colil phage λ receptor”, Proc. Natl. Acad. Sci. USA 89:8651-8655, 1992

Burks, E. A., Chen, G., Georgiou, G., Iverson, B. L. “In vitro Scanning Saturation Mutagenesis of an Antibody Binding Pocket,” Proc. Natl. Acad. Sci. 94:412-7 (1997).

Cadwell and Joyce, PCR Meth. Appl.:2, 28, 1992

Campbell, in: Monoclonal Antibody Technology, Laboratory Techniques in Biochemistry and Molecular Biology, Vol. 13, Burden & Von Knippenberg, Amsterdam, Elseview, pp. 75-83, 1984

Charbit, et al., “Versatility of a vector for expressing foreign polypeptides at the surface of Gram-negative bacteria”, Gene 70:181-189, 1988

Chiswell & Clackson, 1992

Chiswell & McCafferty, “Phage antibodies: will new ‘coliclonal’ antibodies replace monoclonal antibodies?” TIBTECH, 10:80-84, 1992

Chou & Fasman, “Conformational Parameters for Amino Acids in Helical, β-Sheet, and Random Coil Regions Calculated from Proteins,” Biochemistry, 13(2):211-222, 1974b

Chou & Fasman, “Empirical Predictions of Protein Conformation,” Ann. Rev. Biochem., 47:251-276, 1978b

Chou & Fasman, “Prediction of β-Turns,” Biophys. J., 26:367-384. 1979

Chou & Fasman, “Prediction of protein conformation,” Biochemistry, 13(2):222-245, 1974a

Chou & Fasman, “Prediction of the Secondary Structure of Proteins from Their Amino Acid Sequence,” Adv. Enzymol. Relat. Areas Mol. Biol., 47:45-148, 1978a

Clackson et al., “Making antibody fragments using phage display libraries,” Nature, 352:624-628, 1991

Cornellis, et al., “Development of new cloning vectors for the production of immunogenic outer membrane fusion proteins in Escherichia coli.”, Bio/Technology 14:203-208, 1996

Fischetti, V. A., “Gram-positive commensal bacteria deliver antigens to elicit mucosal and systemic immunity”, ASM News 62:405-410, 1996

Francisco et al., “Production and fluorescence-activated cell sorting of Escherichia coli expressing a functional antibody fragment on the external surface,” Proc. Natl. Acad. Sci. USA, 90:10444-10448, 1993

Francisco et al., Proc. Natl. Acad. Sci. USA, 89:2713-2717, 1992

Francisco, Earhart, Georgiou, Proc. Nat'l Acad. Sci. USA. 89:2713-2718, 1992

Francisco, et al., “Transport and anchoring of β-lactamase to the external surface of Escherichia coli.”, Proc. Natl. Acad. Sci. USA 89:2713-2717, 1992.

Francisco, et al., Bio/Technology, 11:491-496, 1993

Frohman, In: PCR Protocols: A Guide to Methods and Applications, Academic Press, N.Y., 1990

Fuchs, et al., “Targeting recombinant antibodies to the surface of Escherichia coli: fusion to a peptido-glycan-associated lipoprotein”, Bio/Technology 9:1369-1372, 1991

Gefter et al., Somatic Cell Genet., 3:231-236, 1977

Georgiou, et al., “Display of heterologous Molecules on the Surface of Microorganisms: From the Screening of Combinatorial Libraries to Live Bacterial Vaccines,” Nature Biotechnol.15:29-35 1997.

Georgiou, et al., “Practical applications of engineering Gram-negative bacterial cell surfaces”, Trends Biotechnol. 11:6-10, 1993.

Georgiou, G. et al, “folding and Aggregation of TEM β-lactamase: Analogies with the Formation of Inclusion Bodies in Escherichia coli”, Protein Science, 3:1953-60, 1994.

Georgiou, Stephens, Stathopoulos, Poetschle, Mendenhall, Earhart, Protein Engineer., 9:239-247, 1996.

Goding, in: Monoclonal Antibodies: Principles and Practice, 2nd Ed., Orlando, Fla., Academic Press pp. 60-61, 65-66, 71-74, 1986

Hansson, et al., “Exression of recombinant proteins on the surface of the coagulase-negative bacterium Staphylococcus xylosus”, J. Bacteriol. 174:4239-4245, 1992

Harlow & Lane (Eds.), Antibodies: A Laboratory Manual, Cold Spring Harbor Laboratory Press, Cold Spring Harbor, N.Y., 1988

Helmann and Chamberlain, “Structure and function of bacterial sigma factors.” Ann. Rev. Biochem. 57: 839-872, 1988.

Ho et al., 1989

Hofnung, M., “Expression of foreign polypeptides at the Escherichia coli cell surface”, Methods Cell Biol. 34:77-105, 1991

Hollfelder, Kirby, Tawfik, Nature, 383:60-63, 1996

Huse et al., “Generation of A Large Combinatorial Library of the Immunoglobulin Repertoire in Phage Lambda,” Science, 246:1275-1281, 1989

Huston et al., 1989

Huston et al., Methods in Enzymology, “Molecular Design and Modeling: Concepts and Applications,” Abelson & Simon, Academic Press, Inc., San Diego, pp. 46-99, 1991

Huston, et. al., “Protein engineering of antibody binding sites: Recovery of specific activity in an anti-digoxin single-chain Fv analogue produced in Escherichia coli,” Proc. Natl. Acad. Sci. USA, 85:5879-5883, 1988

Innis et al., PCR Protocols, Academic Press, Inc., San Diego Calif., 1990

Iverson et al., “A Combinatorial System for Cloning and Expressing the Catalytic Antibody Repertoire in Escherichia coli,” Cold Spring Harbor Symposia on Quantitative Biology, 54:273-281, 1989

Jameson & Wolf, “The Antigenic Index: A Novel Algorithm for Predicting Antigenic Determinants,” Comput. Appl. Biosci, 4(1):181-186, 1988

Janda, et al., J. Am. Chem. Soc., 110:835-4837, 1988

Jeffrey, P. D., Strong, R. K., Sieker, L. C., Chang, C. Y., Campbell, R. L., Petsko, G. A., Haber, E., Margolies, M. N. & Sheriff, S. 26-10 Fab-digoxin complex: affinity and specificity due to surface complementarity. Proc. Nat'l Acad. of Sci. 90, 10310-4 1993.

Jose, et al., “Common structural features of IgA1 protease-like outer membrane protein”, Mol. Microbiol. 18:378-380, 1995

Kabat et al., Sequences of Proteins of Immunological Interest, U.S. Dept. of Health and Human Services, U.S. Government Printing Office, 1987

Kang et al., “Linkage of recognition and replication functions by assembling combinatorial antibody Fab libraries along phage surfaces,” Proc. Natl. Acad. Sci. USA, 88:4363-4366, 1991

Klauser, et al., “Extracellular transport of cholera toxin B subunit using Neisseria IgA protease β-domain: conformation-dependent outer membrane translocation”, EMBO J. 9:1991-1999, 1990

Klauser, et al., “The secretion pathway of IgA protease-type proteins in Gram-negative bacteria”, Bioessays 15:799-805, 1993

Kohler & Milstein, Eur. J. Immunol., 6:511-519, 1976

Kohler & Milstein, Nature, 256:495-497, 1975

Kornacker & Pugsley, Mol. Microbiol, 4:1101-1109, 1990

Kornacker, et al., “The normally periplasmic enzyme β-lactamase is specifically and efficiently translocated through the Escherichia coli outer membrane when it is fused to the cell-surface enzyme pullulanase”, Mol. Microbiol. 4:1101-1109, 1990

Kuby, J. “Immunology”, W. H. Freeman and Co., New York (1990)

Kwoh et al., “Transcription-based amplification system and detection of amplified human immunodeficiency virus type 1 with a bead-based sandwich hybridization format,” Proc. Nat. Acad. Sci. USA, 86:1173-1177, 1989

Laukkanen, et al., “Lipid-tagged antibodies: bacterial expression and characterization of a lipoprotein-single-chain antibody fusion protein”, Protein Engineer 6:449-454, 1993

Lerner, Benkovic, Schultz, Science, 252:659-667, 1991

Liad, MvKenzie, Hageman, Proc. Nat'l Acad. Sci. USA, 83:576-580, 1986

Lilley and Higgins, “Local DNA topology and gene expression: the case of the leu-500 promoter.” Mol. Microbiol. 5, 779-783.

Lu, et al., “Expression of thioredoxin random peptide libraries on the Escherichia coli cell surface as functional fusions to flagellin: A system designed for exploring protein-protein interactions”, Bio/Technology 13:366-372, 1995

Lugtenberg & Van Alphen, “Molecular architecture and functioning of the outer membrane of Escherichia coli and other gram-negative bacteria,” Biochem. Biophys. Acta, 737:51-115, 1983

Makrides S C. Microbiol. Rev. Vol. 60(3):512-538, 1996.

Mangel, Toledo, Brown, Worzalla, Lee, Dunn, Methods Enzymol, 244:384-399, 1994

Maniatis et al., Molecular Cloning: A Laboratory Manual, Cold Springs Harbor Laboratories, Cold Springs Harbor, N.Y., 1982

Maniatis et al., Molecular Cloning: A Laboratory Manual, Cold Springs Harbor Laboratories, Cold Springs Harbor, N.Y., 1989

Matthews, Adv. Prot. Chem., 46:249-278, 1995.

McCafferty et al., “Phage antibodies: filamentous phage displaying antibody variable domains,” Nature, 348:552-554, 1990

Moore and Arnold, Nature Biotechnol., 14:458-467, 1996

Morrison & Desrosiers, “A PCR-Based Strategy for Extensive Mutagenesis of a Target DNA Sequence,” BioTechniques, 14:454457, 1993

Mullinax et al., “Identification of a human antibody fragment clones specific for tetanus toxoid in a bacteriophage λ immunoexpression library,” Proc. Natl. Acad. Sci. USA, 87:8095-8099, 1990

Nakamura et al., Enzyme Immunoassays: Heterogeneous and Homogeneous Systems, Ch. 27, 1987

Newton, et al., “Expression and immunogenicity of an 18-residue epitope of HIV1 gp41 inserted in the flagellar protein of a Salmonella live vaccine, Res. Microbiol. 146: 203-216

Newton, et al., “Topology of the membrane protein LamB by epitope tagging and a comparison with the X-ray model”, J. Bacteriol. 178:3447-3456, 1996

O'Callaghan, et al., “Immunogenicity of foreign peptide epitopes expressed in bacterial envelope proteins”, Res. Microbiol. 141:963-969, 1990

O'Hara, et al., “One-sided polymerase chain reaction: The amplification of cDNA,” Proc. Nat'l Acad. Sci. USA, 86:5673-5677, 1989

Orlandi et al., “Cloning immunoglobulin variable domains for expression by the polymerase chain reaction,” Proc. Nat. Acad. Sci. USA, 86:3833-3837, 1989

Osuna, Flores, Sobeon, Crit. Rev. Microbiol., 20:107-116, 1994

Pallesen, et. al., “Chimeric FimH adhesin of type 1 fimbriae: a bacterial surface display system for heterologous sequences”, Microbology 141:2839-2848, 1995

Palzkill et al., “Selection of functional signal peptide cleavage sites from a library of random sequences.” J Bacteriol, 176 (3) p563-8, 1994

Parmley & Smith, “Antibody-selectable filamentous fd phage vectors: affinity purification of target genes,” Gene, 73:305-318, 1988

Persson et al., “Generation of diverse high-affinity human monoclonal antibodies by repertoire cloning,” Proc. Natl. Acad. Sci. USA, 88:2432-2436, 1991

Petrosino and Palzkill, J. Bacteriol., 178:1821-1828, 1996

Pozzi, et al., “Delivery and expression of a heterologous antigen on the surface of streptococci”, Infect. Immun. 60:1902-1907, 1992

Provence, et al., “Analysis of the extracellular secretion of the putative hemagglutinin Tsh of an avian pathogenic Escherchia coli strain: evidence for an autonomous IgA protease-type secretion”, In Preparation, 1997

Salmong, G. P. C. and Reeves, P. J., “Membrane traffic wardens and protein secretion in Gram-negative bacteria”, Trends Biol. Sci. 18:7-12, 1993

Samuelson, et al., “Cell surface display of recombinant proteins on Staphylococcus carnosus”, J. Bacteriol. 177:1470-1476, 1995

Sastry et al., “Cloning of the immunological repertoire in Escherichia coli for generation of monoclonal catalytic antibodies: Construction of a heavy chain variable region-specific cDNA library,” Proc. Natl. Acad. Sci. USA, 86:5728-5732, 1989

Schildbach et al., Protein Science, 2:206-214, 1993

Schreuder, et al., “Immobilizing proteins on the surface of yeast cells”, Trends Biotechnol. 14:115-120, 1996

Schreuder, et al., “Yeast expressing hepatitis B virus surface antigen determinants on its surface: implications for a possible oral vaccine”, Vaccine 14:383-388, 1996

Schreuder, et. al., 37 Targeting of a heterologous protein to the cell wall of Saccharomyces cerevisiae”, Yeast 9:399-409, 1993

Scott & Smith, “Searching for Peptide Ligands with an Epitope Library,” Science, 249:386-390, 1990

Skerra & Plückthun, “Assembly of a Functional Immunoglobulin Fᵥ Fragment in Escherichia coli,” Science, 240:1038-1041, 1988

Smiley and Benkovic, “Selection of catalytic antibodies for a biosynthetic reaction from a combinatorial cDNA library by complementation of an auxotrophic Escherichia coli: antibodies for orotate decarboxylation.” Proc Nat'l Acad Sci., 91 (18) p8319-23, 1994.

Smith “Surface presentation of protein epitopes using bacteriophage expression systems.” Curr. Opin. Biotechnol. 2 (5) p668-73, 1991., 1991

Smith et al., 1992

Sousa, et al., “Enhanced metalloadsorption of bacterial cells displaying poly-His peptides”, Nature Biotech. 14:1017-1020, 1996

St. Geme, et al., “A Haemophilus influenzae IgA protease-like protein promotes intimate interaction with human epithelial cells”, Mol. Microbiol. 14:217-233, 1994

Stathopoulos et al., Appl. Microbiol. Biotechnol. 45:112-119, 1996

Stathopoulos, et al., “Characterization of Escherichia coli expressing an Lpp'OmpA(46-159)-PhoA fusion protein localized in the outer membrane”, Appl. Microbiol. Biotechnol. 45:112-119, 1996

Steidler, et al., “Pap pill as a vector system for surface for surface exposition of an immunoglobulin G-binding domain of protein A of Staphylococcus aureus in Escherichia coli.”, J. Bacteriol. 175:7639-7643, 1993

Stemmer, Nature, 370-389-391, 1994

Stemmer, Proc. Nat'l Acad. Sci. USA, 91:10747-10751, 1994

Su, et al., “Construction of stable LamB-Shiga toxin B subunit hybrids: analysis of expression in Salmonella typhimurium aroA strains and stimulation of B subunit-specific mucosal and serum antibody responses”, Infect. Immun. 60:3345-3359, 1992

Suzuki, et al., “Extracellular transport of VirG protein in Shigella”, J. Biol. Chem. 270:30874-30880, 1995

Tang, Hicks, Hilvert, Proc. Nat'l Acad. Sci. USA, 88:8784-8786, 1991

Tanke & van der Keur, Trends Biotechnol., 11:55-62, 1992

Tawfik, Green, Chap, Sela, Eshhar, Proc. Nat'l Acad. Sci. USA, 90:373-377, 1993

Taylor, et al., “The TraT lipoprotein as a vehicle for the transport of foreign antigenic determinants to the cell surface of Escherichia coli K12: structure-function relationship in the TraT protein”, Mol. Microbiol. 4:1259-1268, 1990

van Die, et al., “Expression of foreign epitopes in P-fimbriae of Escherichia coli.”, Mol. Gen. Genet. 222:297-303, 1990

Venkatachalam, Huang, Larocco, Palzkill, J. Biol. Chem., 269:23444-23450, 1994

Walker et al., “Isothermal in vitro amplification of DNA by a restriction enzyme/DNA polymerase system,” Proc. Nat'l Acad. Sci. USA, 89:392-396, 1992

White, Chen, Kenyon, Babbitt, J. Biol. Chem., 270:12990-12994, 1995

Winter & Milstein, “Man-made antibodies,” Nature, 349:293-299, 1991

Wolf et al., “An Integrated Family of Amino Acid Sequence Analysis Programs,” Comput. Appl. Biosci., 4(1):187-191, 1988

Wong, et al., “Pseudomonas aeruginosa outer membrane protein OprF as an expression vector for foreign epitopes: the effects of positioning and length on the antigenicity of the epitope”, Gene 158:55-60, 1995

Wu et al., “The Ligation Amplification Reaction (LAR)—Amplification of Specific DNA Sequences Using Sequential Rounds of Template-Dependent Ligation,” Genomics, 4:560-569, 1989

Yang et al., “Electrochemiluminescence: A New Diagnostic and Research Tool,” Bio/Technology, 12:193-194, 1994

Yu and Arnold, Protein. Eng., 9:77-83, 1996.

What is claimed is:

1. A method for selecting a polypeptide from a plurality of candidate proteins, the method comprising the steps of:
(a) obtaining a library of vectors that encode a plurality of distinct candidate polypeptides, wherein said vector provides for the cell surface expression of said candidate polypeptides;
(b) expressing each of said plurality of candidate polypeptides on the surface of a host cell; and
(c) selecting a host cell that expresses a desired polypeptide.

2. The method of claim 1, wherein said host cell is a Gram negative bacterium.

3. The method of claim 2, wherein said host cell is E. coli.

4. The method of claim 1, wherein said polypeptide is selected from the group consisting of an antibody or antibody fragment, an enzyme, a cytokine, a transcription factor, a clotting factor, a chelating agent, a hormone and a receptor.

5. The method of claim 4, wherein said polypeptide is an antibody or antibody fragment.

6. The method of claim 5, wherein selecting a host cell that expresses a desired antibody comprises the steps of:
(a) contacting said antibody- or antibody fragment-expressing cells with a selected antigen; and
(b) identifying a host cell that binds to said selected antigen.

7. The method of claim 6, wherein the antigen is labeled.

8. The method of claim 7, wherein the label is a fluorescent or chemilluminescent label.

9. The method of claim 6, wherein said selected antigen is located on the surface of a cell other than said host cell, and said host cell that binds to said selected antigen is identified by a method comprising the steps of:
(a) contacting said host cell with said cell expressing or having conjugated thereto said selected antigen; and
(b) identifying a host cell bound to said cell expressing or having conjugate thereto said selected antigen.

10. The method of claim 9, further comprising size sorting of bound cells following the step of contacting said host cell with said cell expressing or having conjugated thereto said selected antigen.

11. The method of claim 6, wherein said vector library is obtained by a method comprising the steps of:
(a) administering to an animal an immunologically effective amount of a composition comprising a selected antigen;
(b) obtaining from the animal a plurality of distinct DNA segments that encode distinct antibodies or antibody fragments; and
(c) incorporating said plurality of DNA segments into a plurality of expression vectors, the vectors expressing antibodies or antibody fragments on the outer membrane surface of a Gram negative host cell.

12. The method of claim 11, wherein said plurality of DNA segments are obtained by a method comprising the steps of:
(a) isolating mRNA from antibody-producing cells of said animal;
(b) amplifying a plurality of distinct RNA segments using a set of nucleic acid primers having sequences complementary to antibody constant region or antibody framework region nucleic acid sequences; and
(c) preparing a plurality of distinct DNA segments having sequences complementary to said amplified RNA segments.

13. The method of claim 1, wherein said vector library is obtained by a method comprising the steps of:
(a) obtaining a DNA segment that encodes a selected polypeptide;
(b) mutagenizing said DNA segment to provide a plurality of DNA segments that encode a plurality of polypeptides; and
(c) incorporating said plurality of DNA segments into a plurality of expression vectors, the vectors expressing a plurality of polypeptides on the surface of a Gram negative host cell.

14. The method of claim 13, wherein said polypeptide is an antibody or an antibody fragment.

15. The method of claim 14, wherein said selected cells that express a desired antibody are subjected to cleavage to release the selected antibody or antibody fragment from the surface of the outer membrane.

16. The method of claim 13, wherein selecting a host cell that expresses a desired antibody comprises the steps of:
(a) contacting said antibody- or antibody fragment-expressing cells with a selected antigen; and
(b) identifying a host cell that binds to said selected antigen.

17. The method of claim 16, wherein said selected antigen is linked to a detectable label.

18. The method of claim 17, wherein said selected antigen is linked to a a fluorescent label, a chemilluminescent label, a radioactive label, biotin, avidin, a magnetic bead or an enzyme that generates a colored product upon contact with a chromogenic substrate.

19. The method of claim 18, wherein said cells that bind to said selected antigen are identified by a method comprising the steps of:
(a) contacting said plurality of cells with said detectably labeled antigen under conditions effective to allow specific antigen-antibody binding;
(b) removing non-specifically bound antigen from said cells; and
(c) identifying the antibody- or antibody fragment-expressing cells by detecting the presence of the bound detectable label.

20. The method of claim 19, wherein said cells that bind to said selected antigen are identified by a method comprising the steps of:
(c) contacting said plurality of cells with a fluorescently labeled antigen under conditions effective to allow specific antigen-antibody binding;
(d) subjecting said cells to automated cell sorting; and
(e) identifying the desired antibody or antibody fragment by detecting the fluorescently labeled sorted cells.

21. The method of claim 20, wherein said cells are subjected to sorting by flow cytometry.

22. The method of claim 20, wherein said cells are subjected to a first and a second round of automated cell sorting.

23. The method of claim 22, wherein regrowth of sorted cells is conducted between said first and said second rounds of cell sorting.

24. The method of claim 22, wherein said cells are subjected to a third and a fourth round of automated cell sorting.

25. The method of claim 18, wherein said selected antigen is linked to a magnetic bead.

26. The method of claim 25, wherein cells that band said antigen are selected are identified by a method comprising the steps of:
(a) contacting said plurality of cells with said magnetic bead labeled antigen under conditions effective to allow specific antigen-antibody binding;
(b) subjecting said cells to magnetic sorting; and
(c) identifying the desired antibody- or antibody fragment by detecting the magentic bead labeled sorted cells.

27. The method of claim 4, wherein said polypeptide is an enzyme.

28. The method of claim 27, wherein said cells expressing a desired enzyme are selected on the basis of enzyme activity.

29. The method of claim 28, wherein said enzyme activity is substrate cleavage.

30. The method of claim 29, wherein cleavage of said substrate results in loss of quenching of a detectable signal.

31. The method of claim 28, wherein said enzyme activity is substrate binding.

32. The method of claim 31, wherein said binding results in the quenching of a detectable signal.

33. The method of claim 31, wherein said binding results in the generation of a unique signal not found in the absence of binding.

34. The method of claim 28, wherein said enzyme activity results in the association of a detectable signal with said host cell.

35. The method of claim 34, further comprising sorting said host cell by flow cytometry.

36. The method of claim 35, wherein said cells are subjected to a second round of automated cell sorting.

37. The method of claim 36, wherein regrowth of sorted cells is conducted between said first and said second rounds of cell sorting.

38. A method for catalyzing a chemical reaction, comprising the steps of:
(d) obtaining a host cell that expresses an enzyme or catalytic antibody on the surface of the outer membrane; and
(e) contacting said host cell with a sample containing the necessary substrates for said chemical reaction.


SEQUENCE LISTING (53 sequences)

SEQ ID NO: 1 - DNA, 780 bp; single-stranded, linear
  CDS 1..780
gaagttcaactgcaacagtctggtcctgaattggttaaacctggcgcctctgtgcgcatgtcctgcaaatcctcagggtacattttcaccgacttctacatgaattgggttcgccagtctcatggtaagtctctagactacatcgggtacatttccccatactctggggttaccggctacaaccagaagtttaaaggtaaggccacccttactgtcgacaaatcttcctcaactgcttacatggagctgcgttctttgacctctgaggactccgcggtatactattgcgccggctcctctggtaacaaatgggccatggattattggggtcatggtgctagcgttactgtgagctctggtggcggtggctcgggcggtggtgggtcgggtggcggcggatcagacatagtactgacccagtctccagcttctttggctgtgtctctaggacaaagggccacgatatcctgccgatccagccaaagtctcgtacattctaatggtaatacttatctgaactggtaccaacagaaaccaggacagccacccaagcttctcatctataaggtatccaaccgattctctggagtccctgccaggttcagtggcagtgggtctgagtcagacttcaccctcaccatcgatcctgtggaggaagatgatgctgcaatatattactgtagccaaactacgcatgttccacccacgttcggctcggggaccaagctggagctgaaacgtgctagccagccagaactcgccccggaagaccccgaggac

SEQ ID NO: 2 - protein, 260 aa
EVQLQQSGPELVKPGASVRMSCKSSGYIFTDFYMNWVRQSHGKSLDYIGYISPYSGVTGYNQKFKGKATLTVDKSSSTAYMELRSLTSEDSAVYYCAGSSGNKWAMDYWGHGASVTVSSGGGGSGGGGSGGGGSDIVLTQSPASLAVSLGQRATISCRSSQSLVHSNGNTYLNWYQQKPGQPPKLLIYKVSNRFSGVPARFSGSGSESDFTLTIDPVEEDDAAIYYCSQTTHVPPTFGSGTKLELKRASQPELAPEDPED

SEQ ID NO: 3 - DNA, 18 bp; single-stranded, linear
tggaccaacaacatcggt

SEQ ID NO: 4 - DNA, 26 bp; single-stranded, linear
cccatatcaccagctcaccgtctttc

SEQ ID NO: 5 - DNA, 32 bp; single-stranded, linear
gaccccgaggactaacgtcttcgaataaatac

SEQ ID NO: 6 - DNA, 25 bp; single-stranded, linear
ccgaattcgtttgaacatgcctaac

SEQ ID NO: 7 - DNA, 29 bp; single-stranded, linear
cggaattcgtgcgcaacacgatgaagctc

SEQ ID NO: 8 - DNA, 31 bp; single-stranded, linear
agggcatgcaagggcaccaataactgcctta

SEQ ID NO: 9 - DNA, 26 bp; single-stranded, linear
ttggctgcagtaatatattgcagcat

SEQ ID NO: 10 - DNA, 31 bp; single-stranded, linear
tgcaatatattactgcagccaaactacgcat

SEQ ID NO: 11 - DNA, 30 bp; single-stranded, linear
cggcagtttctgcagatatattcgcaagat

SEQ ID NO: 12 - DNA, 31 bp; single-stranded, linear
cttgcgaatatatctgcagaaactgccggaa

SEQ ID NO: 13 - DNA, 40 bp; single-stranded, linear
acgccacatcttgcgaatatatctgcagaaactgccggaa

SEQ ID NO: 14 - DNA, 18 bp; single-stranded, linear
cagggtacattttcaccg

SEQ ID NO: 15 - DNA, 46 bp; single-stranded, linear
  modified_base one-of(13, 14, 22, 23, 28, 29): /note= “N = A, C, T or G”
  modified_base one-of(15, 24, 30): /note= “B = C, G or T”
aactgcagccaannbacgcatnnbccannbacgttcggctcgggga

SEQ ID NO: 16 - DNA, 63 bp; single-stranded, linear
  modified_base one-of(31, 32, 34, 35, 37, 38, 40, 41): /note= “N = A, C, T or G”
  modified_base one-of(33, 36, 39, 42): /note= “S = C or G”
gtatactattgcgccggctcctctggtaacnnsnnsnnsnnsgattattggggtcatggtgct

SEQ ID NO: 17 - DNA, 30 bp; single-stranded, linear
gttaccagaggagccggcgcaatagtatac

SEQ ID NO: 18 - DNA, 36 bp; single-stranded, linear
tacattttcaccgacttcaatatgaattgggttcgc

SEQ ID NO: 19 - DNA, 36 bp; single-stranded, linear
tacattttcaccgacttctgcatgaattgggttcgc

SEQ ID NO: 20 - DNA, 36 bp; single-stranded, linear
tacattttcaccgacttctctatgaattgggttcgc

SEQ ID NO: 21 - DNA, 36 bp; single-stranded, linear
tacattttcaccgacttcgggatgaattgggttcgc

SEQ ID NO: 22 - DNA, 36 bp; single-stranded, linear
tacattttcaccgacttctaaatgaattgggttcgc

SEQ ID NO: 23 - protein, 9 aa
SQTTHVPPT

SEQ ID NO: 24 - protein, 9 aa
SQATHMPGT

SEQ ID NO: 25 - protein, 9 aa
SQTTHFPVT

SEQ ID NO: 26 - protein, 9 aa
SQATHYPTT

SEQ ID NO: 27 - protein, 9 aa
SQCTHWPVT

SEQ ID NO: 28 - protein, 9 aa
SQTTHVPPT

SEQ ID NO: 29 - protein, 9 aa
SQATHYPST

SEQ ID NO: 30 - protein, 9 aa
SQATHSPST

SEQ ID NO: 31 - protein, 9 aa
SQVTHGPRT

SEQ ID NO: 32 - protein, 9 aa
SQGTHRPYT

SEQ ID NO: 33 - protein, 9 aa
SQITHVPKT

SEQ ID NO: 34 - protein, 9 aa
SQLTHLPRT

SEQ ID NO: 35 - protein, 9 aa
SQPTHVPPT

SEQ ID NO: 36 - protein, 9 aa
SQVTHKPGT

SEQ ID NO: 37 - protein, 9 aa
SQLTHWPST

SEQ ID NO: 38 - protein, 9 aa
SQLTHGPRT

SEQ ID NO: 39 - protein, 9 aa
SQLTHGPRT

SEQ ID NO: 40 - protein, 9 aa
SQZTHGPFT

SEQ ID NO: 41 - protein, 10 aa
SSGNKWAMDY

SEQ ID NO: 42 - protein, 10 aa
SSGNYRALDY

SEQ ID NO: 43 - protein, 10 aa
SSGNRRAWDY

SEQ ID NO: 44 - protein, 10 aa
SSGNRRALDY

SEQ ID NO: 45 - protein, 10 aa
SSGNGRAWDY

SEQ ID NO: 46 - protein, 10 aa
SSGNISALDY

SEQ ID NO: 47 - protein, 10 aa
SSGNQRKMDY

SEQ ID NO: 48 - protein, 9 aa
SQTTHVPPT

SEQ ID NO: 49 - protein, 9 aa
SQVTHRPLT

SEQ ID NO: 50 - protein, 9 aa
SQVTHDPGT

SEQ ID NO: 51 - protein, 9 aa
SQVTHCPST

SEQ ID NO: 52 - protein, 9 aa
SQVTHWPPT

SEQ ID NO: 53 - protein, 9 aa
SQVTHYPVT